(19) United States
(12) Patent Application Publication
(10) Pub. No.: US 2008/0267909 A1
(43) Pub. Date: Oct. 30, 2008

(54) PHAGOCYTE ENHANCEMENT THERAPY FOR ATHEROSCLEROSIS

(75) Inventor: Ira TABAS, New City, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/035,869

(22) Filed: Feb. 22, 2008

Related U.S. Application Data
(63) Continuation-in-part of application No. PCT/US2006/031942, filed on Aug. 16, 2006.
(60) Provisional application No. 60/710,835, filed on Aug. 24, 2005.

(51) Int. Cl. C12Q 1/02; A61K 31/20; A61K 38/17; A61K 38/20; A61K 31/56; A61K 36/06; A61K 31/715; A61K 38/07; A61P 9/10

(52) U.S. Cl. 424/85.2; 435/29; 514/560; 514/12; 514/369; 514/169; 424/195.16; 514/54; 514/18; 514/8

(57) ABSTRACT

Compounds that increase phagocytosis of apoptotic macrophages or necrotic cells that are associated with advanced atherosclerotic lesions are useful for treating atherosclerosis. Methods are provided for identifying such compounds and for preventing or treating atherosclerosis by increasing phagocytosis of apoptotic macrophages associated with advanced atherosclerotic lesions.

### CROSS REFERENCE TO RELATED APPLICATIONS

This application is a continuation in part of PCT/US2006/031942 filed Aug. 16, 2006, which claims the benefit of U.S. Provisional Application 60/710,835, filed Aug. 24, 2005; both of which are hereby incorporated in their entirety by reference.

The U.S. Government may have certain rights in this invention pursuant to Grant No. HL081181-01 awarded by the National Institutes of Health-National Heart, Lung, and Blood Institute.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

One of the early events in atherosclerosis is the entry of monocytes into focal areas of the arterial subendothelium that have accumulated matrix-retained lipoproteins, often including modified lipoproteins. These monocytes differentiate into macrophages and the macrophages accumulate large amounts of intracellular cholesterol through the ingestion of lipoproteins in the subendothelium. The number of these macrophages in lesions provides a measure of atherosclerotic burden. The processes that determine the number of macrophages in a lesion include macrophage proliferation and macrophage depletion, which is determined by macrophage death and macrophage egress from the lesion.

Apoptotic macrophages are more numerous in advanced atherosclerotic lesions compared to early atherosclerotic lesions, suggesting that phagocytic clearance in advanced lesions is defective. The necrotic core of late atherosclerotic lesions is made up primarily of dead macrophages and is rich in inflammatory cytokines. Defective clearance of macrophages is an aspect of late atherosclerotic lesions.

### SUMMARY OF THE INVENTION

The present invention relates to methods of preventing or ameliorating acute cardiovascular clinical events such as atherosclerosis using phagocyte enhancement therapy. Accordingly, the invention relates to a method for treating atherosclerosis or inhibiting the development of atherosclerosis in a subject. The method includes administering to the subject a compound that enhances macrophage phagocytosis.

In one aspect, the invention provides a method for identifying an enhancer of phagocytic clearance of free cholesterol-induced (FC-induced) macrophages, the method comprising (a) labeling an FC-induced apoptotic macrophage; (b) culturing the FC-induced apoptotic macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with FC-induced apoptotic macrophages in the absence of the test compound indicates that the compound is an enhancer of phagocytic clearance of a FC-induced apoptotic macrophages.

In another aspect, the invention provides a method for identifying an enhancer of phagocytic clearance of free cholesterol-induced (FC-induced) macrophages, the method comprising (a) labeling an FC-induced necrotic macrophage; (b) culturing the FC-induced necrotic macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with FC-induced necrotic macrophages in the absence of the test compound indicates that the compound is an enhancer of phagocytic clearance of a FC-induced necrotic macrophages.

In another aspect, the invention provides a method for identifying an enhancer of phagocytic clearance of free cholesterol-loaded macrophages, the method comprising (a) labeling a free cholesterol-loaded macrophage; (b) culturing the free cholesterol-loaded macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with free cholesterol-loaded macrophages in the absence of the test compound indicates that the compound is an enhancer of phagocytic clearance of a free cholesterol-loaded macrophages.

In one embodiment, the free cholesterol-loaded macrophage is a foam cell.

In another embodiment, the phagocytes are derived from peritoneal macrophages.

In still another embodiment, the FC-induced apoptotic macrophage is labeled with calcein-AM.

In another embodiment, the FC-induced apoptotic macrophage is labeled with annexin V.

In yet another embodiment, the FC-induced necrotic macrophage is labeled with annexin V.

In still a further embodiment, the FC-induced necrotic macrophage is labeled with propidium iodide.

In one embodiment, the apoptotic macrophage is induced by contacting the macrophage with acetyl-low density lipoprotein (acetyl-LDL) and an acyl-coenzyme A:cholesterol acyltransferase (ACAT) inhibitor. In another embodiment, the necrotic macrophage is induced by contacting the macrophage with acetyl-low density lipoprotein (acetyl-LDL) and an acyl-coenzyme A:cholesterol acyltransferase (ACAT) inhibitor. In still another embodiment, the cholesterol-loaded macrophage is induced by contacting the macrophage with acetyl-low density lipoprotein (acetyl-LDL) and an acyl-coenzyme A:cholesterol acyltransferase (ACAT) inhibitor. In one embodiment, the ACAT inhibitor is 58035 ACAT inhibitor.

In still a further embodiment, the increase in the amount of label present in the phagocytes of the test sample compared to the amount of label in the macrophages cultured in the absence of the test compound is at least 10%, 20%, 25%, 30%, 50%, 75%, 90%, or 100% greater than the amount of label in the macrophages cultured in the absence of the test compound.

In yet another embodiment, the FC-induced apoptotic macrophages of step (b) are cultured in the presence of a test compound and a statin, and the amount of label present in the phagocytes in the test sample is compared to the amount of label present in the phagocytes in the absence of the test compound and in the presence of the statin.

In one aspect, the invention provides a method for promoting clearance of apoptotic macrophages from advanced atherosclerotic lesions, the method comprising contacting an atherosclerotic lesion with a compound that promotes clearance of apoptotic macrophages. In one embodiment, the method further comprises contacting the atherosclerotic lesion with a statin. In another embodiment, the compound that promotes clearance of apoptotic macrophages is a lipoxin, a lipoxin analog, a compound that stimulates lipoxin synthesis or activity, an annexin-1, an apolipoprotein E, a RhoA inhibitor, a RhoA kinase inhibitor, a thiazolinedione, interleukin-4, interleukin-13, a corticosteroid, eotaxin, yeast cell wall extract, β1-glucan, acemannan, tuftsin, a C1qRp ligand, an activator of 11-beta-hydroxysteroid dehydrogenase, a CCAAT/enhancer binding protein alpha, and inhibitor of farnesylation, an inhibitor of geranylgeranylation, or a compound that inhibits expression or activity of Cdc44.

In another aspect, the invention provides a method for treating atherosclerosis or inhibiting the development of atherosclerosis in a subject, the method comprising administering to the subject a compound that enhances macrophage phagocytosis.

In another aspect, the invention provides a method for treating a subject at risk of having or having an atherosclerotic lesion, the method comprising administering to the subject a pharmaceutically effective amount of a compound that promotes clearance of apoptotic macrophages from advanced atherosclerotic lesions.

In another aspect, the invention provides a method for treating a subject at risk of having or having an atherosclerotic lesion, the method comprising administering to the subject a pharmaceutically effective amount of a compound that promotes clearance of anecrotic macrophages from advanced atherosclerotic lesions.

In another aspect, the invention provides a method for treating a subject at risk of having or having an atherosclerotic lesion, the method comprising administering to the subject a pharmaceutically effective amount of a compound that promotes clearance of cholesterol-loaded macrophages from advanced atherosclerotic lesions.

In one embodiment, a statin is administered to the subject. In another embodiment, the subject is characterized as having a history of heart disease, having diabetes, having atherosclerosis, or any combination thereof.

In some embodiments the compound is an annexin-1, an apolipoprotein E, an interleukin-4, an interleukin-13, a lipoxin, is a thiazolinedione, a yeast cell wall extract, β1-glucan, acemannan, tuftsin, a C1qRp ligand, an activator of 11-beta-hydroxysteroid dehydrogenase, a CCAAT/enhancer binding protein alpha, and inhibitor of farnesylation, an inhibitor of geranylgeranylation, a compound that inhibits expression or activity of Cdc44, an annexin-1 peptidomimetic, an annexin-1 truncation product, an annexin-1 fragment, a lipoxin peptidomimetic, a lipoxin truncation product, a lipoxin fragment, a histidine-rich glycoprotein (HRG), a fragment thereof, or a derivative thereof (e.g., a fragment that includes the N1N2 domain of HRG or mimics the activity of the N1N2 domain), an apoplipoprotein E peptidomimetic, an apoplipoprotein E truncation product, an apoplipoprotein E fragment or a compound that inhibits RhoA or a RhoA kinase. In one embodiment, the compound that inhibits RhoA or a RhoA kinase is fasudil or Y-27632.

In another aspect, the invention provides a composition comprising an enhancer of phagocytic clearance of apoptotic macrophages (a phagocyte enhancer compound) and a pharmaceutically acceptable excipient. In one embodiment, the phagocyte enhancer compound is identified using a method for identifying an enhancer of phagocytic clearance of free cholesterol-induced (FC-induced) macrophages, the method comprising (a) labeling an FC-induced apoptotic macrophage; (b) culturing the FC-induced apoptotic macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with FC-induced apoptotic macrophages in the absence of the test compound indicates that the compound is an enhancer of phagocytic clearance of a FC-induced apoptotic macrophages.

In another aspect, the invention provides a composition comprising an enhancer of phagocytic clearance of apoptotic macrophages (a phagocyte enhancer compound) and a pharmaceutically acceptable excipient. In one embodiment, the phagocyte enhancer compound is identified using a method for identifying an enhancer of phagocytic clearance of free cholesterol-induced (FC-induced) macrophages, the method comprising (a) labeling an FC-induced necrotic macrophage; (b) culturing the FC-induced necrotic macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with FC-induced necrotic macrophages in the absence of the test compound indicates that the compound is an enhancer of phagocytic clearance of a FC-induced necrotic macrophages.

In another aspect, the invention provides a composition comprising an enhancer of phagocytic clearance of apoptotic macrophages (a phagocyte enhancer compound) and a pharmaceutically acceptable excipient. In one embodiment, the phagocyte enhancer compound is identified using a method for identifying an enhancer of phagocytic clearance of free cholesterol-loaded macrophages, the method comprising (a) labeling a free cholesterol-loaded macrophage; (b) culturing the free cholesterol-loaded macrophage in the presence of phagocytes and in the presence of a test compound, thereby providing a test sample; and (c) determining the amount of a label present in the phagocytes in the test sample, wherein, an increase in the amount of the label in the phagocytes in the presence of the test compound compared to the amount of the label present in the phagocytes cultured with free cholesterol-loaded macrophages in the absence of the test compound indicates that the compound is an enhancer of phagocytic clearance of a free cholesterol-loaded macrophages.

In one embodiment, the composition further comprises a statin.

The invention also relates to a composition that includes a phagocyte enhancer compound and a pharmaceutically acceptable excipient, for example, a phagocyte enhancer compound identified using a method described herein. In certain embodiments, the composition also includes a statin. The composition can be provided in a kit, for example, a kit including instructions for use.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE FIGURES

FIG. 1 is a diagram of the events of early atherosclerotic lesion physiology (left) and late atherosclerotic lesion physiology (right).

FIG. 2 is a bar graph depicting the results of experiments assaying ingestion of FC-induced macrophages by phagocytes (i.e., peritoneal macrophages). Data represent triplicate samples +/−SEM and the differences between all three groups were statistically significant p<0.05).

FIG. 3A is a bar graph depicting the results of experiments examining the effect of rosiglitazone (ROSI) and the Rho inhibitor C3 on phagocytosis. Results are presented as the mean +/−SEM. N=3 with each counted field containing approximately 150 cells.

FIG. 3B is a bar graph depicting the results of experiments examining the effect of ROSI and the ROCK kinase inhibitor Y-27632 on phagocytosis. Results are presented as the mean +/−SEM. N=3 with each counted field containing approximately 150 cells.

FIG. 4 is a bar graph depicting the results of experiments in which phagocytes were treated with 10 μM Y-27632 and their ability to ingest apoptotic macrophages compared to untreated controls. Data are expressed as the percent of phagocytes that ingested apoptotic macrophages.

FIG. 5 is a bar graph depicting the results of experiments in which phagocytes were treated with 10 μM fasudil, and their ability to phagocytose apoptotic macrophages compared to untreated controls. Data are expressed as the percentage of phagocytes that ingested apoptotic macrophages.

FIG. 6A is a set of reproductions of micrographs of FC-AMs that were labeled with Calcium Green™-AM (green) and then briefly exposed to phagocytes. Non-ingested FC-AMs were removed by stringent rinsing of the cells and then incubated for 24 hours in fresh medium containing the ACAT inhibitor 58035. The cells were then stained with Alexa Fluor 594-annexin V to detect apoptosis. The left panel is a reproduction of the green-filter image (ingesting phagocytes, or “IPs”), the middle panel is a reproduction of red-filter image (apoptosis), and the right panel is a reproduction of the phase image.

FIG. 6B is a set of reproductions of micrographs of macrophages that were incubated for 18 hour in medium alone (control) or with medium containing 100 μg/ml acetyl-LDL plus 10 μg/ml ACAT inhibitor 58035 to effect FC loading (FC-AMs). The macrophages were then assayed for apoptosis by staining with Alexa Fluor 594-annexin V. Bar, 10 μm.

FIG. 7 is a bar graph depicting the results of experiments in which macrophages were incubated for 18 hours in medium alone (i.e., no exposure to FC-AMs) or in the same medium for the indicated time points after ingestion of FC-AMs. All of the incubations contained [¹⁴C]oleate, and some of the phagocytes were incubated with 1 μM U18666A during the post-ingestion period to block cholesterol trafficking to the endoplasmic reticulum (ER). To make sure that the phagocytes would not be exposed to residual ACAT inhibitor in the FC-AMs, the FC-AMs for this experiment were generated by incubating macrophages from Acat1−/− mice with AcLDL without ACAT inhibitor. After the indicated time points, the macrophages were assayed for cholesteryl [¹⁴C]oleate formation as an indicator of cholesterol trafficking to ACAT in the ER.

FIG. 8A is a bar graph depicting the results of experiments in which FC-AMs were labeled with Alexa Fluor 488-annexin V (green) and then added to phagocytes for 30 minutes. The phagocytes were washed to remove non-ingested FC-AMs and incubated in fresh medium containing ACAT inhibitor for 3 hours. The phagocytes were then subjected to FACS sorting to separate IPs from non-IP macrophages. Lipids were extracted from the IPs or non-IP macrophages, and FC mass was measured was by gas-liquid chromatography. Results are expressed as cellular free cholesterol.

FIG. 8B is a bar graph depicting the FC mass ratio in macrophages incubated for 10 hours in medium containing acetyl-LDL+58035 to effect FC loading (FC-AMs) versus incubation in medium alone. The second bar is the FC mass ratio in IPs chased for 10 hour after ingestion of FC-AMs versus non-IPs.

FIG. 8C is a bar graph depicting the results of experiments in which macrophages were incubated for 10 hours in medium alone or medium containing acetyl-LDL+58035 to effect FC loading (FC-AMs) (First and second bars). The third and fourth bars depict the results of experiments in which macrophages were incubated for either 7 hours or 20 hours post-ingestion of FC-AMs and free cholesterol mass was measured. The results for the third and fourth bars were normalized using the basal level of free cholesterol in control macrophages and the percentage of phagocytes ingesting FC-AMs (22%).

FIG. 8D is a bar graph depicting the results of experiments in which FC-AMs were induced by incubation with [³H]-acetyl-LDL+58035. Phagocytes were then exposed to these FC-AMs and, after non-ingested FC-AMs were removed, chased for 15 minutes or 20 hours in fresh media containing ACAT inhibitor. The media were then collected assayed for tritium radioactivity. The results are expressed as a percent of total tritium (i.e., cells+medium tritium) that was in the medium.

FIG. 9A is a set of reproductions of micrographs of macrophages that were exposed briefly to FC-AMs that had been labeled with Alexa Fluor 488-annexin V (green) and then, after removal of the non-ingested FC-AMs, incubated for 1 hour in fresh medium containing DiI-labeled acetyl-LDL (red). The cells were then viewed for green fluorescence to identify IPs (left panel) and red fluorescence to identify acetyl-LDL uptake (middle panel); the merged image is shown in the right panel.

FIG. 9B is a bar graph depicting the results of experiments determining the amount of cellular free cholesterol in IPs that were incubated in medium containing ACAT inhibitor alone for 3 hours or 20 hours post-FC-AM ingestion (first and second bars). The third bar is the result for IPs incubated for 20 hours post-ingestion in medium containing acetyl-LDL+58035 to effect additional FC-loading. The IPs were isolated by FACS as for those of FIG. 8A and assayed for FC mass.

FIG. 9C is a set of reproductions of micrographs of macrophages that were exposed briefly to FC-AMs that had been labeled with Calcium Green™-AM (green) and then, after removal of the non-ingested FC-AMs, incubated for 20 hours in fresh medium containing acetyl LDL+58035. The cells were then assayed for apoptosis using Alexa Fluor 594-annexin V (red). Panel 1 shows green fluorescence to identify IPs and panel 2 shows red fluorescence to identify apoptosis. The merged image is shown in the third panel, and the phase image is shown in the fourth panel. The fifth panel shows the quantified data for the percent of IPs (green cells) and non-JPs (non-green cells) that were labeled with red annexin V. Bar, 10 μm.

FIG. 10A is a bar graph depicting the results of experiments in which the protocol described in FIG. 9C was used and the percent apoptosis was determined in non-IPs (cross-hatched bars) and IPs (black bars) that were incubated for 20 hours in FC-loading medium either in the absence or presence of 10 μM of the IKK inhibitor PS1145, 10 μM of the PI-3 kinase/Akt inhibitor LY294022, or both compounds.

FIG. 10B is a photographic reproduction of the results of immunoblotting experiments in which macrophages were either exposed or not exposed to FC-AMs and then incubated for the indicated time in medium containing ACAT inhibitor; “c” refers to control macrophages not exposed to FC-AMs and “p” (phagocytosis) refers to macrophages exposed to FC-AMs. Cell lysates were subjected to SDS-PAGE and immunoblotted for phosphorylated AKT and total AKT.

FIG. 11A is a photographic reproduction of immunoblots of Bcl-2 from macrophages from Bcl2^{flox}×LysMCre mice and macrophages from wild type or Bcl2^{flox} mice. Bcl-xL is a control for a closely related member of the Bcl family, and actin is the loading control.

FIG. 11B is a set of reproductions of photomicrographs of Fc-AMs prepared using the protocol of FIG. 9C and labeled with Calcium Green™-AM (green) and then added to phagocytes derived from Bcl2^{flox} mice or Bcl2^{flox}×LysMCre mice. Non-ingested FC-AMs were removed by wash and phagocytes were incubated for 20 hours in fresh medium containing acetyl LDL+58035. The cells were then assayed for apoptosis using Alexa Fluor 594-annexin V (red). The first panel shows fluorescence (green) to identify IPs and panel 2 shows red fluorescence to identify apoptosis. The merged image is shown in the third panel, and the phase image is shown in the fourth panel. Bar, 10 μm.

FIG. 11C is a bar graph depicting the quantified data for the percent of IPs (green cells) vs. non-IPs (non-green cells) that were labeled with red annexin V.

FIG. 12 is a bar graph depicting the results of experiments in which, using the protocol of FIG. 9C, FC-AMs were labeled with Calcium Green™-AM and then added to phagocytes for 30 minutes. The phagocytes were washed to remove non-ingested FC-AMs, incubated in fresh medium for 10 minutes, and then subjected to UV irradiation for 20 min. After an additional 8 hour incubation in medium alone or containing 10 μM of the IKK inhibitor PS1145 or 10 μM of the PI-3 kinase/Akt inhibitor LY294022, the cells were assayed for apoptosis using Alexa Fluor 594-annexin V. Shown are the quantified data for the percent of non-JPs (cross-hatched bars) and IPs (black bars) that were labeled with annexin V.

FIG. 13. Total body weight and plasma lipoproteins of Apoe−/− and MertkKD;Apoe−/− mice. Body weight, total plasma cholesterol, and fast performance liquid chromatography gel-filtration profiles (from pooled plasma samples) from male Apoe−/− and MertkKD;Apoe−/− mice fed a Western-type diet for either (A) 10 wks or (B) 16 wks. n.s., statistically non-significant difference between the two groups of mice.

FIG. 14. Aortic root lesion area in Apoe−/− and MertkKD;Apoe−/− mice after 10 wks and 16 wks on a Western-type diet. The images show two examples each of sections of hematoxylin and eosin-stained aortic roots from each group of mice fed a Western-type diet for either (A) 10 wks or (B) 16 wks. Bar, 0.3 mm. Below each set of sections are graphs showing the quantified lesion area. n.s., statistically non-significant difference between the two groups of mice.

FIG. 15. TUNEL-positive nuclei are increased in the aortic root lesions of MertkKD;Apoe−/− mice. TUNEL analysis of aortic root sections from Apoe−/− and Mertk−/−;Apoe−/− mice fed a Western-type diet for either (A) 10 wks or (B) 16 wks. Micrographs display Hoechst-stained nuclei (blue), TUNEL-positive signal (red), and the merged images. Bar, 100 μm. Quantified data are shown below the images (* indicates p<0.05).

FIG. 16. Plaque necrosis is increased in the aortic root lesions of MertkKD;Apoe−/− mice after 16 wks on a Western-type diet. The images show examples of sections of hematoxylin and eosin-stained aortic roots from Apoe−/− and Mertk−/−;Apoe−/− mice fed a Western-type diet for 16 wks. nec, necrotic areas. Bar, 100 μm. The graph shows quantification of necrotic areas (n=10 per group; * indicates p<0.05).

FIG. 17. Primary efferocytes were pre-treated with the indicated cytokine for about 24 hours and then challenged for 30 minutes with UV-irradiated apoptotic J774 cells and the percent efferocytosis was determined.

### DETAILED DESCRIPTION OF THE INVENTION

The issued patents, applications, and other publications that are cited herein are hereby incorporated by reference to the same extent as if each was specifically and individually indicated to be incorporated by reference.

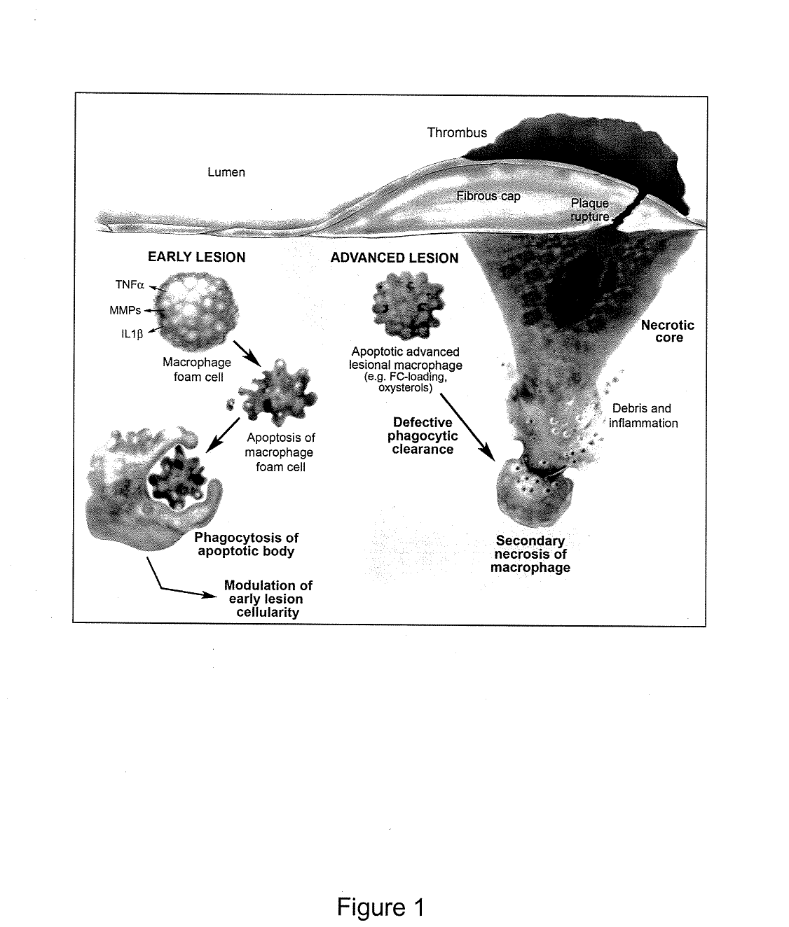

Late phase atherosclerotic events include an accumulation of apoptotic cells in association with atherosclerotic lesions. In early atherogenesis, apoptotic macrophages associated with atherogenic lesions are rapidly cleared by phagocytic macrophages. Living foam cells (lipid-laden macrophages) play a pro-atherogenic role by secreting cytokines and other molecules, and the net effect of macrophage apoptosis in early lesions is modulation of lesion cellularity and decreased lesion progression (FIG. 1, right). In late lesions, macrophages also undergo apoptosis, but phagocytic clearance of these apoptotic macrophages is not efficient and secondary necrosis of the apoptotic macrophages occurs. This contributes to the generation of the necrotic core feature of an advanced lesion. In turn, this promotes inflammation, plaque instability, and acute lesional thrombosis. Residual surviving macrophages also play a role in promoting the progression of advanced lesions.

The present invention relates to a method of preventing or treating advanced atherosclerosis by enhancing phagocytic activity associated with late atherosclerotic lesions (advanced atherosclerotic plaques), thereby decreasing the number and rate of accumulation of apoptotic and necrotic cells associated with late atherosclerotic lesions. In the method, one or more compounds that can enhance phagocytosis of apoptotic cells, necrotic cells, or both are administered to a subject having or at risk for advanced atherosclerosis.

It is a fundamental property of phagocytes that they selectively recognize and ingest apoptotic cells. Therefore, treatment or prevention of atherosclerosis (e.g., advanced atherosclerosis) can be effected by enhancing phagocyte activity of phagocytes associated with advanced atherosclerotic lesions. Accordingly, methods are described herein for identifying compounds that increase phagocytosis and are effective for increasing phagocytosis in advanced atherosclerotic lesions. Also described herein are methods of using such compounds, e.g., for prevention or treatment of atherosclerosis.

Atherosclerotic lesions can lead to various types of ischemia, including, but not limited to, ischemia of the heart, brain, or extremities, and can result in infarction. The earliest type of lesion (e.g., a fatty streak) is an inflammatory lesion, consisting of monocyte-derived macrophages and T-lymphocytes. In persons with hypercholesterolemia, the influx of these cells is preceded by the extracellular deposition of amorphous and membranous lipids.

The cells of most organs and tissues satisfy their requirements for membrane cholesterol via endogenous cholesterol biosynthesis. Many cell types, have acquired mechanisms to internalize exogenous sources of cholesterol, usually in the form of plasma-derived lipoproteins. Examples include steroid-synthesizing cells, hepatocytes, and macrophages. A portion of the cholesterol in such cells is either synthesized de novo, internalized as lipoprotein or taken up as VLDL or LDL via the LDL receptor. Some of this cholesterol can originate from cell detritus ingested during phagocytosis and from chemically modified lipoproteins taken up via cell surface receptors. Cells that internalize exogenous cholesterol also repress endogenous cholesterol biosynthesis and LDL receptor expression in response to cholesterol loading. Cells also have mechanisms to prevent the accumulation of excess unesterified, or “free,” cholesterol (FC). One mechanism is cholesterol esterification, which is mediated by the microsomal enzyme acyl-coenzyme A:cholesterol acyltransferase (ACAT). The two forms, ACAT-1 and ACAT-2, differ in their sites of expression, with macrophages and most other cell types expressing ACAT-1. In humans, intestinal epithelial cells, but not hepatocytes, selectively express ACAT-2; mice, in contrast, express ACAT-2 in both of these cell types. Another important protective mechanism against FC accumulation is cellular efflux of cholesterol and certain cholesterol-derived oxysterols. Cholesterol laden macrophage foam cells can be found in atherosclerotic lesions. Foam cells can further exacerbate the atherosclerotic lesion by secreting cytokines to attract other effectors of the immune response, e.g., neutrophils.

Although cells can adapt to FC loading, prolonged internalization can cause these mechanisms to fail, leading to cell death. FC-loaded macrophages show signs of both necrosis (e.g., disrupted cell membranes) and apoptosis (e.g., condensed nuclei) and some cells in a population of FC-loaded macrophages become necrotic, whereas others undergo a programmed apoptotic response. Moreover, cells that initially undergo an apoptotic program can subsequently demonstrate morphological signs of necrosis (aponecrosis). This can occur as a result of chronic ATP depletion or failure of neighboring cells to phagocytose the apoptotic bodies. Thus, in a cell-culture model of FC-loaded macrophages, all or only a subpopulation cells may show the apoptosis-associated hallmarks of phosphatidylserine externalization and DNA fragmentation. Similarly a cell-culture model of FC-loaded macrophages, all or only a subpopulation cells may show the necrosis-associated hallmarks loss of membrane integrity and release of intracellular contents.

FC loading of macrophages can result in several biochemical and morphological changes including, for example, cytosolic and nuclear condensation. With more prolonged FC loading, however, the macrophages can demonstrate signs more characteristic of necrosis, such as swelling of organelles and disruption of the plasma membrane. In some cases, FC-loaded macrophages can be condensed into “apoptotic bodies,” and be phagocytosed by neighboring macrophage without release of cellular contents. In other cases, apoptosis can precede necrosis and does not always prevent release of cellular contents from dying cells. In intact cells, phosphatidylserine is restricted to the inner leaflet of the plasma membrane. However, during early apoptosis before the loss of membrane integrity, phosphatidylserine appears on the outer leaflet of the plasma membrane. Thus, early apoptotic cells interact with the phosphatidylserine-binding protein, annexin V. The membrane-impermeable nucleic acid stain propidium iodide (PI) is excluded by early apoptotic cells but stains necrotic cells and cells undergoing late apoptosis (J Histochem Cytochem. 1998 August; 46(8):895-900). For example, FC-loaded macrophages can be cultured in the presence Annexin that is conjugated to a fluorescent label or tag. Annexin V-fluorescein (FITC) is widely employed in cytometry and microscopy as an early marker for apoptosis because of its binding affinity for phosphatidilserine (PS), which is exposed at the cell surface early in apoptosis (Martin et al. 1995 J Exp Med 182:1545-1556; Reutelingsperger and van Heerde, 1997 Cell Mol Life Sci 53:527-532).

Calcein-AM is a vital dye that is useful for detection and tracking of apoptosis in living cells by confocal laser microscopy (Bussolati et al. 1995, Exp Cell Res 220:283-291). This neutral vital dye is loaded and rapidly converted by cell esterases into its negative, impermeant fluorescent analogue. The nucleus-cytoplasm signal intensity ratio is approximately 3:1 and allows clear visualization of both structures. In cells undergoing apoptosis, early chromatin condensation is read as a sharp nuclear signal increase, and initial cell shrinkage is also visualized. When chromatin begins to fragment and eventually is segregated within blebs, the process can be tracked stepwise in real time. At the same time, it is possible to check membrane integrity, whose preservation is one of the most significant features of apoptosis with respect to necrosis, because in the presence of membrane defects calcein leaks out of the cell and the signal also vanishes in the presence of residual esterase activity (Morris 1990, Bio Techn 8:296-312; Weston and Parish 1990, J Immunol Methods 133:87-97).

### Assays for Enhancers of Phagocytosis of Macrophages

The invention provides methods (also referred to herein as “screening assays”) for identifying modulators (e.g., enhancers) of phagocytosis associated with atherosclerotic lesions. The modulators can include proteins, peptides, peptidomimetics, peptoids, small molecules including small non-nucleic acid organic molecules and small inorganic molecules, nucleic acids such as antisense nucleic acids, siRNAs, or other oligonucleotide molecules, or other drugs. To identify enhancers of phagocytosis of macrophages (phagocyte enhances), a compound is tested in one or more assays related to detecting the ability of the compound to increase phagocytosis of macrophages, e.g., using macrophages having one or more characteristics of macrophages associated with late atherosclerotic lesions. In general, the macrophages have at least one, two, or more features of apoptosis such as caspase activation, DNA fragmentation, annexin V staining, and condensed nuclei. Methods of identifying these features are known in the art.

In one example of an in vitro assay, FC-induced macrophages are labeled and co-cultured with phagocytes in the presence or absence of a test compound. After incubation for an amount of time sufficient to permit phagocytosis of labeled FC-induced macrophages, the cultures are washed to removed unphagocytized FC-macrophages and the amount of label present in the phagocytes is determined. An increase in the number of phagocytes that have ingested labeled apoptotic cells and/or an increase in the amount of label per phagocyte cultured in the presence of the test compound compared to those cultured in the absence of the test compound (a control) indicates that the test compound is a candidate compound for increasing phagocytosis of apoptotic macrophages associated with advanced atherosclerotic lesions.

In one aspect, the invention relates to a method of identifying an enhancer of phagocytic clearance of apoptotic macrophages. The method includes labeling an apoptotic macrophage, culturing the apoptotic macrophage in the presence of phagocytes in the presence of a test compound, thereby providing a test sample, and determining the amount of label present in the phagocytes in the test sample, such that, an increase in the amount of label in the phagocytes in the presence of the test compound compared to the amount of label present in the phagocytes in the absence of the test compound indicates that the compound is an enhancer of phagocytic clearance of apoptotic macrophages.

The invention also relates to a method of identifying an enhancer of phagocytic clearance of necrotic macrophages. The method includes labeling an necrotic cell, culturing the necrotic cell in the presence of phagocytes in the presence of a test compound, thereby providing a test sample, and determining the amount of label present in the phagocytes in the test sample, such that, an increase in the amount of label in the phagocytes in the presence of the test compound compared to the amount of label present in the phagocytes in the absence of the test compound indicates that the compound is an enhancer of phagocytic clearance of necrotic cells.

The invention also relates to a method of identifying an enhancer of phagocytic clearance of free cholesterol loaded macrophages. The method includes labeling an free cholesterol loaded cell, culturing the free cholesterol loaded cell in the presence of phagocytes in the presence of a test compound, thereby providing a test sample, and determining the amount of label present in the phagocytes in the test sample, such that, an increase in the amount of label in the phagocytes in the presence of the test compound compared to the amount of label present in the phagocytes in the absence of the test compound indicates that the compound is an enhancer of phagocytic clearance of free cholesterol loaded cells.

FC-induced macrophages can be prepared using methods known in the art. Exemplary method include, but are not limited to methods described in Yao and Tabas (2000, J. Biol. Chem. 275:23807-23813) and Mori et al. (2001, J. Lipid Res. 42:1771-1781). In the first method, macrophages are incubated with acetyl-LDL plus an inhibitor of the cholesterol esterifying enzyme acyl-coenzyme A-cholesterol acyltransferase (ACAT). In the second method, activated macrophages are exposed to atherogenic lipoproteins followed by lipoprotein withdrawal. These cells (FC-induced macrophages or FC-induced apoptotic macrophages) are suitable for use in assays described herein to identify compounds that increase phagocytosis of such cells. For use in assays, the FC-induced macrophages are labeled with a molecule that can be transferred to a phagocyte when an FC-induced macrophage is ingested. Labels include vital dyes such as fluorescently labeled annexin-V, calcein-AM, and octadecylrhodamine. In some cases, the macrophages that are FC-loaded to generate FC-induced apoptotic macrophages are from the same source as the macrophages that are used as phagocytes.

In one embodiment, FC-apoptotic macrophages can be macrophages that are undergoing or have undergone early apoptosis and have not yet undergone late apoptosis or undergone necrosis. In another embodiment, FC-apoptotic macrophages can be macrophages that have are undergoing or have undergone late apoptosis and have not undergone necrosis. In another embodiment, FC-induced apoptotic macrophages can be macrophages that are undergoing or have undergone necrosis.

The phagocytes used in this type of assay are derived from, for example, peritoneal macrophages that are harvested from an animal by peritoneal lavage. Phagocytes can be identified using methods known in the art, for example using markers such as those described in Cook et al. (2003, J. Immunol. 171(9):4816-4823).

When a labeled FC-induced apoptotic macrophage is phagocytized, the label (e.g., a vital dye) is internalized and can be detected. The amount of label transferred to the phagocytes is determined, for example, by counting the percentage of phagocytes that have accumulated label, and provides a measure of the amount of phagocytosis in the assay. Methods of assaying the amount of label transferred are known in the art and include, in the case of a dye, flow cytometry, fluorescent microscopy, including high-throughput fluorescent microscopy, or a fluorescent plate reader. The amount of label can be compared to a reference, e.g., a control. In general, the amount of label transferred is determined after a period of time sufficient for phagocytosis to occur. The amount of time required can be determined empirically, but is generally 30 minutes to 60 minutes. The amount of label that is transferred can be, e.g., about 5%, 10%, 20%, 30%, 50%, 75%, 90%, or 100%. In general, the number of FC-induced macrophages used in an assay is greater than the number of phagocytes used in the assay, for example a ratio of about 5:1 FC-induced macrophages:phagocytes. In some embodiments of the assay, the phagocytes are labeled with a dye or other molecule that can be distinguished from the FC-induced apoptotic cell label. For example, the FC-induced macrophages are labeled with a red label such as Red Fluorescent Protein (RFP) and the phagocytes are labeled with Green Fluorescent Protein (GFP). In another non-limiting example, FC-induced macrophages are induced with calcein-AM and phagocytes are labeled with octdecylrhodamine. Phagocytes that have ingested material from FC-induced macrophages can be distinguished by their color, for example, using fluorescence microscopy. In such assays, the number of such cells is counted. An increase in the number of cells that have ingested FC-induced macrophages in the presence of a test compound is compared to a control. For example, in the absence of a test compound, after incubation for about 30 minutes, the level of ingested cells in such an assay is about 15%. An increase in the percentage of ingested cells in the presence of a test compound indicates that the test compound is useful for increasing phagocytosis of FC-induced macrophages.

In some cases, acetyl-low density lipoprotein (acetyl-LDL) and an acyl-coenzyme A:cholesterol acyltransferase (ACAT) inhibitor are used to generate FC-induced apoptotic macrophages. Non-limiting examples of ACAT inhibitors are 58035 (Sandoz Pharmaceutical Corp., East Hanover, N.J.), F1394 (Fujirebio, Malvern, Pa.), CI-976 (Parke-Davis, Morris Plains, N.J.), and CP-113818 (Pfizer, Inc., Groton, Conn.), or PD-138142-15 (Parke-Davis).

Other methods known in the art for generating a system of phagocytes and apoptotic macrophages can be used for the screens using the general method described herein.

As indicated herein, in certain methods to test a compound for its ability to modulate phagocytosis of FC-induced macrophages, a test compound is added to the assay system containing both FC-induced apoptotic macrophages and uninduced/fresh macrophages (i.e., phagocytes). After incubation for a suitable amount of time, the culture plates are rinsed to remove FC-induced macrophages that were not phagocytized and the uninduced macrophages are assayed for label. The number of phagocytes that have ingested FC-induced macrophages is determined. Alternatively, a change in the amount of dye in a sample incubated with the test compound compared to a control sample (i.e., a corresponding sample that was not incubated with the test compound) indicates that the test compound modulates phagocyte activity. For example, a compound that increases the amount of dye in the uninduced macrophages compared to a control is a compound that increases phagocytosis (e.g., of FC-induced apoptotic macrophages). Such compounds are candidate compounds for preventing or treating atherosclerosis. A compound that decreases the amount of dye in the uninduced macrophages compared to a control is a compound that decreases phagocytosis.

Compounds that increase the amount of one or more receptors associated with phagocytosis of apoptotic cells are useful for increasing phagocytosis. One example of such a receptor is the receptor tyrosine kinase MerTK (Mertk). MerTK is a necrotic macrophage receptor that can mediate apoptotic cell clearance of apoptotic thymocytes (Scott et al., 2001, Nature 411:207-211). It has been found that the MerTK receptor is also important for the ingestion of free-cholesterol induced apoptotic macrophages. The amount of MerTK receptor can be assayed using methods known in the art including immunocytochemical methods using an antibody to detect MerTK receptor (e.g., anti-human MerTK (sc-6872), Santa Cruz Biotechnology, Santa Cruz, Calif.) using, for example, an enzyme-linked immunosorbent assay (ELISA) format or using flow cytometry. Compounds that increase MerTK can be assayed, for example, by contacting cells that can express a MerTK in the presence and absence of a test compound. The cells are tested for the expression of MerTK poly A⁺ RNA, expression of MerTK protein, or MerTK activity. A compound that can increase the amount of MerTK expression or activity is a candidate compound for increasing phagocytosis, particularly, phagocytosis associated with advanced atherosclerotic lesions.

In another approach to identifying compounds that are useful for enhancing activity of phagocytes that are associated with advanced atherosclerotic lesions, compounds are tested for their ability to increase the survival of phagocytes that are associated with advanced atherosclerotic lesions. This can be accomplished by, for example, blocking FC trafficking to the endoplasmic reticulum (ER) (e.g., see U.S. Patent Application No. 20040259853 for general methods that can be adapted for us in tests using phagocytes). Survival of phagocytes can be determined by assaying for the absence of phagocyte death using methods known in the art such as by measuring annexin-V staining, TUNEL staining, or active caspase staining. Phagocyte survival is tested in the presence and absence of a compound. Compounds that increase cell survival are candidate compounds for enhancing phagocyte activity.

Yet another approach to identifying compounds that can enhance phagocyte activity is to test compounds for their ability to increase cholesterol efflux from phagocytes. For example, compounds identified as described in U.S. Patent Application No. 20030235878 can be tested for their ability to enhance phagocyte activity.

Following apoptosis, secondary necrosis of macrophages can occur in advanced atherosclerotic lesions. Necrotic cell death is characterized by the rapid and disorganized swelling and rupture of the cell. A necrotic-like cell death pathway has also been identified (e.g., Proskuryaov et al., 2003, Exp. Cell Res. 283:1-16; Kitanaka et al., 1999, Cell Death Differ. 6:508-515). Accordingly, compounds that enhance phagocytosis of necrotic cells are useful for preventing or treating advanced atherosclerosis. For example, compounds that increase expression or activity of histidine-rich glycoprotein (HRG) or a fragment thereof (such as a fragment that includes the N1N2 domain of HRG) that is active in promoting phagocytosis of necrotic cells are useful for enhancing phagocytosis of necrotic cells associated with advanced atherosclerotic lesions. An example of an assay that can be used to identify compounds that promote enhanced phagocyte activity with respect to necrotic cells, is similar to the assay described herein, in which labeled macrophages (phagocytes) are incubated with FC-induced macrophages that are labeled such that they can be distinguished from the phagocytes. However, necrotic cells are used instead of FC-induced macrophages. An example of such an assay is found in Jones et al. (2005, J. Biol. Chem., 280:35733-35741). A test compound is included in a sample containing both macrophages and necrotic cells and an increase in the number of necrotic cells phagocytized by macrophages in the presence of the test compound compared to a control indicates that the compound enhances phagocytosis of necrotic cells.

Further, in vivo assays can also be conducted to determine whether a compound is effective for increasing phagocytosis of macrophages, e.g., macrophages associated with late atherosclerotic lesions. For example, an animal model of atherosclerosis can be treated with a compound and examined for size and stage of atherosclerotic lesions, macrophage content of advanced lesions, number of apoptotic macrophages, extent of lesional necrosis, inflammatory cytokines, thinning or rupture of the fibrous cap, and thrombosis or other features of atherosclerotic lesions. The treated animals are compared to untreated controls. A compound that decreases an undesirable feature, e.g. of advanced atherosclerosis is useful for treating atherosclerosis. Animal models for atherosclerosis are known in the art, for example, apoE^{-/-} mice and LDL receptor deficient mice (Jackson Laboratories, Bar Harbor, Me.) (See, Smith et al., 1997, J. Intern. Med. 242:99-109). A suitable non-human primate can be used such as the model using cynomolgus monkeys that is described in Kitamoto et al. (2004, Arterioscler. Thromb. Vasc. Biol. 24(8):1522-8). Such in vivo assays are generally conducted using compounds identified as enhancers of phagocytosis in in vitro assays.

Other methods useful for enhancing phagocyte activity include increasing the activity of specific proteins that have been identified as promoting phagocyte activity. Such proteins include annexin 1, lipoxin, interleukin-4 (IL-4) and interleukin-13 (IL-13). Methods useful for increasing the activity of a protein are known in the art and include introducing a sequence that can express such a protein in a cell e.g., using recombinant nucleic acid methods, or contacting a cell with a compound that activates a pathway that includes stimulation of the protein.

Methods of increasing the amount of a receptor for apoptotic macrophages on a phagocyte are also useful for enhancing phagocyte activity. Such receptors are known in the art, for example, see Henson et al. (2001, Curr. Biol. 11:R795-R805) and Savill et al. (2000, Nature 407:784-788).

It has been reported that certain molecules induced by or otherwise affected by glucocorticoids are associated with increased phagocytosis (Giles et al., 2001, J. Immunol. 167(2):976-86). Accordingly, methods of increasing the expression or activity of such molecules associated with reported glucocorticoids induction of phagocytosis are also useful for enhancing phagocytosis associated with advanced atherosclerosis. Such compounds exclude glucocorticoids and other compounds that are glucocorticoid mimetics. Examples include recruitment of paxillin and pyk2 to focal contacts and a down-regulation of p130Cas. Also, compounds that increase levels of active Rac and cytoskeletal activity can be useful in the methods.

### Compounds

Compounds useful in the invention (phagocytosis enhancers) include compounds identified using methods described herein. Compounds that can be useful for enhancing phagocyte activity associated with advanced atherosclerotic lesions include lipoxin, a lipoxin analog (e.g., see U.S. Pat. No. 6,831,186; 15-epi-16-parafluoro-LXA4), or a compound that stimulates lipoxin synthesis or activity such as adenosine 3′5′-cyclic monophosphorothioate, Rp-isomer, triethylammonium salt (Rp-cAMP; Godson et al., 2003, J. Immunol. 164:1663-1667), an apolipoprotein, annexin-I or a biologically active fragment thereof, an annexin-I analog, other compounds used for treatment of autoimmune disorders, a pentarphin such as a cyclopentarphin (see, U.S. Patent Application Publication No. 20050143293), yeast cell wall extract, β1 glucan (e.g., U.S. Pat. No. 5,786,343), acemannan (see, U.S. Pat. No. 5,106,616), tuftsin (Najjar et al., 1970, Nature 228:672-673); C1qRp ligands (e.g., U.S. Pat. No. 5,965,439), IL-4, IL-13, a compound that enhances IL-3 production (e.g., a corticosteroid) (J Immunol. 1997 Jun. 15; 158(12):5589-95), a compound that enhances IL-13 production (e.g., eotaxin) (Gastroenterology, Volume 127, Issue 1, Pages 105-118), a compound that induced alternative activation of macrophages (see Nat Rev Immunol. 2003 January; 3(1):23-35), or a compound that that can reduce oxidative stress in a cell. Additional phagocytosis enhancers useful for increasing phagocytosis of apoptotic macrophages (e.g., for treating or preventing cardiovascular disease) include, without limitation, an activator of 11-beta-hydroxysteroid dehydrogenase (e.g., forskolin (Rubis et al., 2004, Acta Biochim. Pol. 51(4):919-924), CCAAT/enhancer binding protein alpha (C/EBPalpha; Apostolova et al., 2005, Am. J. Physiol. Endocrinol. Metab. 288(5):E957-964), an inhibitor of farnesylation (AZD3409; Appels et al., 2006, Anal. Chem. 15; 78(8):2617-2622), ABT-100 (Fong et al., 2006, Science 311(5767):1621-1623), FTI-277 (Efuet et al., 2006, Cancer Res. 66(2):1040-1051), an inhibitor of geranylgeranylation (e.g., a farnesyl transferase inhibitor (AZD3409, GGTI-298 (Efuet et al., 2006, Cancer Res. 66(2):1040-1051), and a RhoA inhibitor including an inhibitor of RhoA kinase (ROCK). RhoA signaling inhibitors useful for increasing phagocytosis of apoptotic macrophages (e.g., for treating or preventing cardiovascular disease) include the bacterial C3 exoenzyme that ribosylates Rho, the ROCK inhibitor Y-27632 (which selectively targets p160ROCK; Sigma-Aldrich, St. Louis, Mo.), H-1152 (EMD Biosciences, San Diego, Calif.) and fasudil (USBio, Swampscott, Mass.).

Thiazolinendiones (TZDs) are a class of drugs that signal through the transcription factor, PPAR-gamma, and can enhance phagocytosis of apoptotic macrophages. Non-limiting examples of TZDs that are useful for enhancing phagocytosis of apoptotic macrophages (e.g., to treat or prevent cardiovascular disease) include ciglitazone, troglitazone (Rezulin), rosiglitazone (Avandia™) and pioglitazone (Actos), a selective peroxisome proliferator-activated receptor (PPAR) modulator (SPPARM), a selective PPARgamma modulator, a glitazone (a dual PPAR activator) such as a compound that activates both alpha and gamma PPAR isoforms, e.g., Galida (tesaglitazar; AstraZenica) and muraglitazar (Bristol-Meyers Squibb). Small molecule inhibitors of Rho kinase can also be used (e.g., small molecules identified by BioAxone Therapeutic Inc., Montreal, Canada). Dominant negative genetic approaches can also be used to effect enhancement of phagocytosis, e.g., by making constructs for Rho, Rac, Cdc42 that inhibit expression of activity. Methods of making such constructs are known in the art.

Another class of compounds useful as phagocyte enhancers are compounds that inhibit the expression or activity of CD44, e.g., antibodies directed against CD44 (for example, Hart et al., J. Immunol. 1997, 159:919-925).

The test compounds of the invention can also be obtained using any of the numerous approaches in combinatorial library methods known in the art, including: biological libraries; peptoid libraries (libraries of molecules having the functionalities of peptides, but with a novel, non-peptide backbone that are resistant to enzymatic degradation but that nevertheless remain bioactive; see, e.g., Zuckermann et al. (1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993, Proc. Natl. Acad. Sci. U.S.A. 90:6909), Erb et al. (1994, Proc. Natl. Acad. Sci. USA 91:11422), Zuckermann et al. (1994, J. Med. Chem. 37:2678) Cho et al. (1993, Science 261:1303), Carrell et al. (1994, Angew. Chem. Int. Ed. Engl. 33:2059), Carell et al. (1994, Angew. Chem. Int. Ed. Engl. 33:2061), and in Gallop et al. (1994, J. Med. Chem. 37:1233).

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner, U.S. Pat. No. 5,223,409), spores (Ladner U.S. Pat. No. 5,223,409), plasmids (Cull et al. (1992, Proc. Natl. Acad. Sci. USA 89:1865-1869) or on phage (Scott and Smith (1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310; Ladner).

In some cases, a compound of the invention interferes with the activity of a molecule (and is referred to as an inhibitory compound) that inhibits phagocyte activity (referred to as a phagocyte inhibitory molecule). Examples of such compounds include inhibitors of RhoA and Rho kinase (Tosello-Trampont et al., 2003, J. Biol. Chem. 278(50):49911-49919). Such inhibitors are useful for enhancing phagocyte activity associated with atherosclerosis. Such inhibitory compounds can include, for example, an isolated nucleic acid molecule that is antisense to a nucleic acid corresponding to an inhibitory molecule. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire coding strand, or to only a portion thereof. In some cases, the antisense nucleic acid molecule is antisense to a noncoding region of the coding strand of a nucleotide sequence (e.g., the 5′ or 3′ untranslated regions).

As discussed above, compounds can also be identified that enhance phagocyte activity associated with necrotic cells that are associated with advanced lesions. Such compounds include compounds that increase the expression an activity of HRG, including fragments containing the N1N2 region of HRG.

An antisense nucleic acid can be designed such that it is complementary to the entire coding region of mRNA encoding an inhibitory molecule, but generally is an oligonucleotide that is antisense to only a portion of the coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid that is useful as described herein can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules are typically administered to a subject (e.g., by direct injection at a tissue site), or generated in situ from nucleic acid constructs that can express such molecules. The antisense nucleic acid molecules can hybridize with, or bind to, cellular mRNA and/or genomic DNA encoding an inhibitory molecule to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens and are then internalized. The antisense nucleic acid molecules can also be delivered to cells using the vectors described herein and using methods known in the art. To achieve sufficient intracellular concentrations of the antisense molecules, vector constructs in which the antisense nucleic acid molecule are generally placed under the control of a strong pol II or pol III promoter.

In another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

In still another embodiment, an antisense nucleic acid of the invention is a ribozyme. A ribozyme having specificity for an inhibitory molecule encoding nucleic acid can include one or more sequences complementary to the nucleotide sequence of the inhibitory molecule and a sequence having known catalytic sequence responsible for mRNA cleavage (see U.S. Pat. No. 5,093,246 or Haselhoff and Gerlach, 1988, Nature 334:585-591). For example, a derivative of a Tetrahymena L-19 IVS RNA can be constructed in which the nucleotide sequence of the active site is complementary to the nucleotide sequence to be cleaved in an inhibitory molecule-encoding mRNA. See, e.g., Cech et al. U.S. Pat. No. 4,987,071; and Cech et al. U.S. Pat. No. 5,116,742. Alternatively, mRNA can be used to select a catalytic RNA having a specific ribonuclease activity from a pool of RNA molecules. See, e.g., Bartel and Szostak, 1993, Science 261:1411-1418.

Gene expression of an inhibitory molecule can be inhibited by targeting nucleotide sequences complementary to the regulatory region of the sequence encoding the molecule (e.g., the promoter and/or enhancers) to form triple helical structures that prevent transcription of the gene in target cells. See generally, Helene, 1991, Anticancer Drug Des. 6:569-84; Helene, 1992, Ann. N.Y. Acad. Sci. 660:27-36; and Maher, 1992, Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

A nucleic acid molecule used to inhibit expression of an inhibitory molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al., 1996, Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al., 1996; Perry-O'Keefe et al. 1996, Proc. Natl. Acad. Sci. 93: 14670-14675.

PNAs of nucleic acid molecules corresponding to sequences encoding an inhibitory molecule can be used in therapeutic and diagnostic applications. For example, PNAs can be used as antisense or antigene agents for sequence-specific modulation of gene expression by, for example, inducing transcription or translation arrest or inhibiting replication. PNAs of nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al., 1996, supra)); or as probes or primers for DNA sequencing or hybridization (Hyrup B. et al., 1996, supra; Perry-O'Keefe et al., supra).

In other embodiments, the oligonucleotide (e.g., antisense nucleic acid or expression vector that can express such a molecule) can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al., 1988, Bio-Techniques 6:958-976) or intercalating agents. (see, e.g., Zon, 1988, Pharm. Res. 5:539-549). To this end, the oligonucleotide can be conjugated to another molecule, (e.g., a peptide, hybridization triggered cross-linking agent, transport agent, or hybridization-triggered cleavage agent).

RNA interference (RNAi) is a process whereby double-stranded RNA (dsRNA) induces the sequence-specific degradation of homologous mRNA in animals and plant cells (Hutvagner and Zamore, 2002, Curr. Opin. Genet. Dev. 12:225-232; Sharp, 2001, Genes Dev. 15:485-490). In mammalian cells, RNAi can be triggered by, e.g., approximately 21-nucleotide (nt) duplexes of small interfering RNA (siRNA) (Chiu et al., 2002, Mol. Cell. 10:549-561; Elbashir et al., 2001, Nature 411:494-498), or by micro-RNAs (miRNA), functional small-hairpin RNA (shRNA), or other dsRNAs which are expressed in vivo using DNA templates with RNA polymerase III promoters (Zeng et al., 2002, Mol. Cell. 9:1327-1333; Paddison et al., 2002, Genes Dev., 16:948-958; Lee et al., 2002, Nature Biotechnol. 20:500-505; Paul et al., 2002, Nature Biotechnol. 20:505-508; Tuschl, 2002, Nature Biotechnol. 20:440-448; Yu et al., 2002, Proc. Natl. Acad. Sci. USA, 99:6047-6052; McManus et al., 2002, RNA 8:842-850; Sui et al., 2002, Proc. Natl. Acad. Sci. USA 99:5515-5520).

Examples of molecules that can be used to decrease expression of an inhibitory molecule include double-stranded RNA (dsRNA) molecules that can function as siRNAs targeting nucleic acids encoding the inhibitory molecule and that comprise 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, e.g., a transcribed region of a nucleic acid and the other strand is identical or substantially identical to the first strand. The dsRNA molecules can be chemically synthesized, or can be transcribed in vitro from a DNA template, or in vivo from an engineered RNA precursor, e.g., shRNA. The dsRNA molecules may be designed using methods known in the art (e.g., “The siRNA User Guide,” available at rockefeller.edu/labheads/tuschl/siRNA) and can be obtained from commercial sources, e.g., Dharmacon, Inc. (Lafayette, Colo.) and Ambion, Inc. (Austin, Tex.).

Negative control siRNAs generally have the same nucleotide composition as the selected siRNA, but without significant sequence complementarity to the targeted genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence. Such negative controls are used to, e.g., confirm the specificity of a test siRNA.

The siRNAs for use as described herein can be delivered to a cell by methods known in the art and as described herein in using methods such as transfection utilizing commercially available kits and reagents. Viral infection, e.g., using a lentivirus vector can be used.

An siRNA or other oligonucleotide can also be introduced into the cell by transfection with an heterologous target gene using carrier compositions such as liposomes, which are known in the art, e.g. Lipofectamine™ 2000 (Invitrogen, Carlsbad, Calif.) as described by the manufacturer for adherent cell lines. Transfection of dsRNA oligonucleotides for targeting endogenous genes can be carried out using Oligofectamine™ (Invitrogen, Carlsbad, Calif.). The effectiveness of the oligonucleotide can be assessed by any of a number of assays following introduction of the oligonucleotide into a cell. These assays include, but are not limited to, Western blot analysis using antibodies that recognize the targeted gene product following sufficient time for turnover of the endogenous pool after new protein synthesis is repressed, and Northern blot analysis to determine the level of existing target mRNA.

Still further compositions, methods and applications of RNAi technology for use as described herein are provided in U.S. Pat. Nos. 6,278,039, 5,723,750 and 5,244,805, which are incorporated herein by reference.

### Pharmaceutical Compositions

The compounds described herein and identified using methods described herein that are useful for preventing or treating atherosclerosis by enhancing activity of phagocytes associated with advanced atherosclerotic lesions can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, inhalation, transdermal (topical), transmucosal, and rectal administration; or oral. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an agent that delays absorption, for example, aluminum monostearate or gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle that contains a basic dispersion medium and other ingredients selected from those enumerated above or others known in the art. In the case of sterile powders for the preparation of sterile injectable solutions, the methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the selected pharmaceutical carrier.

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, it is generally desirable to design a delivery system that targets such compounds to the focal site of the disease, e.g., atherosclerotic lesions, to minimize potential damage to unaffected cells are tissues, thereby reducing side effects.

The data obtained from the cell culture assays and animal studies can be used in formulating a range of dosage for use in humans. The dosage of such compounds generally lies within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, about 0.01 to 25 mg/kg body weight, about 0.1 to 20 mg/kg body weight, about 1 to 10 mg/kg, about 2 to 9 mg/kg, about 3 to 8 mg/kg, about 4 to 7 mg/kg, or about 5 to 6 mg/kg body weight. The protein or polypeptide can be administered one time per week for between about 1 to 10 weeks, for example, between 2 to 8 weeks, between about 3 to 7 weeks, about 4, 5, or 6 weeks, or chronically. The skilled artisan will appreciate that certain factors may influence the dosage and timing to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

For antibodies, the dosage is generally 0.1 mg/kg of body weight (for example, 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration (e.g., into the brain). A method for lipidation of antibodies is described in Cruikshank et al. (1997, J. Acquired Immune Deficiency Syndromes and Human Retrovirology 14:193).

In general, a compound that can enhance phagocytosis associated with advanced atherosclerotic lesions is administered to a high-risk subject in an acute or semi-acute setting to stabilize their plaques (lesions). The subject can then be maintained on the compound for a sufficient time to allow the plaque-stabilizing effects of a simultaneously administered cholesterol-lowering drug to become manifest, for example, for about one to two years or longer.

The present invention encompasses compounds that modulate phagocytosis associated with advanced atherosclerotic lesions. A compound can, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The compounds described herein can be conjugated to another moiety such as an antibody, for example, for targeting the compound for delivery to advanced atherosclerotic lesions.

Nucleic acid molecules that are identified for use as compounds useful for enhancing phagocytic activity as described herein can be inserted into vectors and used as gene therapy vectors. Gene therapy vectors can be delivered to a subject by, for example, intravenous injection, local administration (see, U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is embedded. Alternatively, where the complete gene delivery vector can be produced intact from recombinant cells, e.g., retroviral vectors, the pharmaceutical preparation can include one or more cells which produce the gene delivery system. Other methods of delivery of nucleic acids as gene therapy vectors that are known in the art can also be used. Such methods can be combined with other targeted delivery methods such as a stent.

Compounds that are effective for increasing phagocytosis of apoptotic macrophages associated with atherosclerotic lesions, can be modified for targeting to atherosclerotic lesions or delivered using methods that provide them more directly to a lesion. For example, a compound can be delivered to a site identified as containing atherosclerotic lesions using a drug delivery stent. Drug-delivery stents are known in the art (for example, see U.S. Pat. Nos. 6,918,929; 6,758,859; 6,899,729; and 6,904,658), and can be adapted to deliver compounds that enhance phagocytosis, including compounds identified using the methods described herein.

In some embodiments, a pharmaceutical composition includes a statin with a phagocyte enhancer molecule. The phagocytic enhancer molecule can have an effect that is additive to the statin with respect to a therapeutic effect (e.g., for increasing phagocytic clearance of apoptotic macrophages), synergistic to the statin with respect to a therapeutic effect of the statin such as an anti-inflammatory effect and/or LDL-cholesterol lowering effect (e.g., increasing phagocytic clearance of apoptotic macrophages), or increase the therapeutic effect of the statin by countering an adverse effect that the statin has on phagocytic clearance of macrophages. Any therapeutic strategy based on phagocytosis enhancement should be additive to or synergistic with statin therapy if it is to be used with such therapy. The pharmaceutical compositions can be included in a container, pack, or dispenser, and can be provided in a kit with instructions for administration.

### Methods of Treatment

Provided herein are both prophylactic and therapeutic methods of treating a subject at risk of (or susceptible to) having atherosclerosis, in particular, advanced atherosclerosis, characterized by having advanced atherosclerotic lesions. As used herein, the term “treatment” is defined as the application or administration of a therapeutic agent to a subject (e.g., a non-human mammal or a human) in need thereof with the purpose to cure, heal, alleviate, relieve, alter, remedy, ameliorate, improve or affect the disease, the symptoms of disease or the predisposition toward disease. Subjects include, for example, individuals having at least one of a history of heart disease, diabetes, arteriosclerosis, hypercholesterolemia, hypertension, cigarette smoking, obesity, metabolic syndrome, physical inactivity or other disorders or symptoms associated with atherosclerosis (e.g., see The Merck Manual Sixteenth Edition, Berkow, ed., Merck Research Laboratories, Rahway, N.J., 1992). A therapeutic agent includes, but is not limited to, small molecules, peptides, antibodies, ribozymes, antisense oligonucleotides, siRNA and other compounds described herein.

The invention provides a method for preventing in a subject a disease or condition associated with insufficient phagocytosis associated with advanced atherosclerotic lesions by administering to the subject a compound that enhances the activity of phagocytes associated with advanced atherosclerotic lesions. The compound can enhance phagocytosis of apoptotic cells associated with advanced atherosclerotic lesions, phagocytosis of necrotic cells associated with advanced atherosclerotic lesions, or both. Subjects at risk for having advanced atherosclerotic lesions can be identified by methods known in the art, which can include angiography, ultrasound, CT scan, or other indicia of atherosclerosis. In addition, symptoms of atherosclerosis such as critical stenosis, thrombosis, aneurysm, embolus, decreased blood flow to a tissue, angina on exertion, bruit can be used to identify a subject having or at risk for atherosclerosis. Administration of a prophylactic agent can occur prior to the manifestation of symptoms characteristic of having atherosclerosis or advanced atherosclerotic lesions such that disease or disorder is prevented or, alternatively, delayed in its progression.

As discussed herein, compounds, e.g., an agent identified using an assay described above, that exhibits the ability to enhance phagocytosis, particularly phagocytosis associated with advanced atherosclerotic lesions, can be used in accordance with prevention or treatment methods described herein to prevent and/or ameliorate symptoms of atherosclerosis. Such molecules can include, but are not limited to peptides, phosphopeptides, peptoids, small non-nucleic acid organic molecules, inorganic molecules, and proteins including, for example, antibodies (e.g., polyclonal, monoclonal, humanized, anti-idiotypic, chimeric or single chain antibodies, and Fab, F(ab′)₂ and Fab expression library fragments, scFV molecules, and epitope-binding fragments thereof).

Further, oligonucleotides including antisense, siRNA and ribozyme molecules that inhibit expression of a gene whose product inhibits phagocytosis can also be used in accordance with the invention to increase the level of phagocytosis. Still further, triple helix molecules can be utilized in reducing the level of activity of such a gene product. Antisense, ribozyme and triple helix molecules are discussed above. In some cases, compounds that increase the expression, and thereby the activity of a gene product that is associated with increased phagocytosis are used in a method for preventing or treating atherosclerosis. In such cases, nucleic acid molecules that encode and express such gene products (polypeptides) are introduced into cells via gene therapy methods. In some cases, precursor cells for phagocytes (e.g., monocytes) are obtained, in general from the subject to be treated, and the precursor cells are subjected ex vivo to gene therapy to introduce the desired nucleic acid sequence encoding a polypeptide or a regulatory nucleic acid sequence that is introduced into the genome of the phagocyte precursor cell in such a way that it promotes expression of an endogenous gene that increases phagocyte activity. The precursor cell is then introduced into the subject as a treatment method.

Another method by which nucleic acid molecules are utilized in treating or preventing atherosclerosis is through the use of aptamer molecules specific for a protein that, when contacted by a binding partner, promotes phagocytosis, e.g., in advanced atherosclerotic lesions. Aptamers are nucleic acid molecules having a tertiary structure that permits them to specifically bind to protein ligands (see, e.g., Osborne, et al., 1997, Curr. Opin. Chem. Biol. 1:5-9; and Patel, 1997, Curr. Opin. Chem. Biol. 1:32-46). Since nucleic acid molecules may in many cases be more conveniently introduced into target cells than therapeutic protein molecules may be, aptamers offer a method by which phagocytosis can be specifically enhanced without the introduction of drugs or other molecules that may have pluripotent effects.

Antibodies or biologically active fragments thereof that are useful as compounds for enhancing phagocytosis associated with atherosclerosis can be generated and identified using methods known in the art. Such antibodies or fragments can be administered to a subject to treat or prevent atherosclerosis.

In instances where the target antigen is intracellular and whole antibodies are used, internalizing antibodies can be used. Lipofectin™ or liposomes can be used to deliver the antibody or a fragment of the Fab region that binds to the target antigen into cells. Where fragments of the antibody are used, the smallest inhibitory fragment that binds to the target antigen is generally used. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993, Proc. Natl. Acad. Sci. USA 90:7889-7893).

The identified compounds that increase phagocytosis in advanced atherosclerotic lesions as described herein can be administered to a subject at therapeutically effective doses to prevent, treat or ameliorate atherosclerosis. A therapeutically effective dose refers to that amount of the compound sufficient to result in amelioration of at least one symptom of the disorder. Toxicity and therapeutic efficacy of such compounds can be determined by standard pharmaceutical procedures known in the art.

Data obtained from the cell culture assays and animal studies can be used in formulating a range of dosage for use in humans. The dosage of such compounds generally lies within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to increase phagocytosis associated with advanced atherosclerotic lesions is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell et al. (1996, Curr. Opin. Biotechnol. 7:89-94) and in Shea (1994, Trends Polymer Sci. 2:166-173. Such “imprinted” affinity matrixes are amenable to ligand-binding assays, whereby the immobilized monoclonal antibody component is replaced by an appropriately imprinted matrix. An example of the use of such matrices in this way can be seen in Vlatakis et al. (1993, Nature 361:645-647). Through the use of isotope labeling, the “free” concentration of compound that increases phagocytosis can be monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiber optic devices, in turn allowing the dose in a test subject to be quickly optimized based on its individual IC₅₀. A rudimentary example of such a “biosensor” is discussed in Kriz et al. (1995, Analytical Chemistry 67:2142-2144).

Combinations of compounds can be used to prevent or treat atherosclerosis using at least one compound described herein or identified using methods described herein. Such combinations can include, e.g., two or more compounds that increase phagocytosis associated with advanced atherosclerotic lesions or at least one compound that increases phagocytosis and at least one compound useful for treating atherosclerosis whose method of function is unknown or does not directly relate to increasing phagocytic activity associated with advanced atherosclerotic lesions. In one example, the combination includes a compound that is an enhancer of phagocytosis and a compound that can act as an inhibitor of death (e.g., apoptosis) of macrophages associated with advanced atherosclerotic lesions. In another example, at least one compound is administered that can enhance phagocytosis of apoptotic cells associated with advanced atherosclerotic lesions and at least one compound that can enhance phagocytosis of necrotic cells associated with advanced atherosclerotic lesions.

The phagocyte enhancer compounds described herein can be used in the preparation of a medicament for use in the treatment of atherosclerosis, e.g., atherosclerosis associated with advanced atherosclerotic lesions that can be ameliorated using a compound that increases phagocytosis associated with such lesions.

The following examples illustrate the present invention, and are set forth to aid in the understanding of the invention, and should not be construed to limit in any way the scope of the invention as defined in the claims which follow thereafter.

### EXAMPLES

The invention is further illustrated by the following examples. The example is provided for illustrative purposes only. They are not to be construed as limiting the scope or content of the invention in any way.

### Example 1

### Enhancement of Phagocytosis of FC-Induced Macrophages

Enhancers of phagocytosis can work by promoting actin rearrangement through inhibition of protein kinase A (PKA). In advanced atherosclerosis, the goal is to enhance the phagocytosis of apoptotic macrophages, many of which become susceptible to apoptosis in association with loading of free cholesterol (FC). Experiments were conducted to test whether an enhancer of phagocytosis in inflammation can enhance phagocytosis of FC-induced apoptotic macrophages by macrophage phagocytes. Briefly, mouse peritoneal macrophages were labeled with the fluorophore calcein-AM (green) and then FC-loaded to induced apoptosis. Some of the macrophages were not FC-loaded, thus serving as a non-apoptotic control. The macrophages were added to a monolayer of octadecylrhodamine-labeled (red) macrophage phagocytes for 30 minutes at 37° C. The monolayers were then thoroughly rinsed with phosphate buffered saline (PBS). In one condition, phagocytes were pre-treated with 100 μM adenosine 3′,5′-cyclic monophosphorothioate, Rp-isomer, triethylammonium salt (Rp-cAMP; Calbiochem/EMD Biosciences, San Diego, Calif.) for 15 minutes prior to their exposure to apoptotic macrophages. The percentage of rhodamine-labeled phagocytes with green inclusion was determined and quantified. Inclusion of green indicated the uptake of apoptotic cells into the phagocytes.

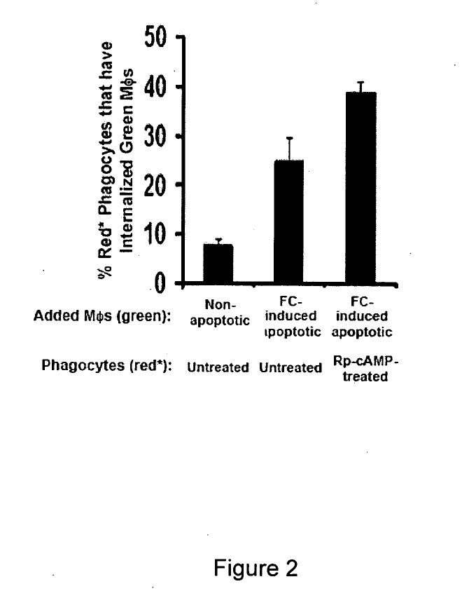

It was found that phagocytes internalized significantly more FC-induced apoptotic macrophages compared to non-apoptotic macrophages (FIG. 2, first two bars of the graph). Phagocytes treated with the PKA inhibitor internalized more FC-induced apoptotic macrophages than untreated phagocytes (compare the second and third bars of FIG. 2).

These data demonstrate that a compound that can enhance the ability of phagocytes to ingest apoptotic cells in other systems can be applied to the phagocytic clearance of FC-induced apoptotic macrophages. These data therefore indicate that phagocytic enhancers can be used to promote the clearance of apoptotic macrophages in advanced atherosclerosis, thereby reducing or preventing lesional necrosis, plaque disruption, acute atherothrombotic clinical events, and other phenomena associated with advanced atherosclerotic lesions.

### Example 2

### Enhancement of Phagocytosis of Apoptotic Macrophages Using Thiazolinendiones (TZDs)

Thiazolinendiones (TZDs) are a class of drugs that signal through the transcription factor (PPAR-gamma). Experiments were performed demonstrating that TZDs can enhance phagocytosis, and likely function by inhibition of RhoA, which signals through Rho kinase (ROCK). Briefly, in these experiments, peritoneal macrophages were cultured in L-cell conditioned medium and treated with 10 μM rosiglitazone (ROSI, a TZD) in dimethylsulfoxide (DMSO) or treated with DMSO alone (CTRL) for 18 hours. Subsequently, the ROCK kinase inhibitor Y-27632 or C3 (Clostridium botulinum C3 exoenzyme (an inhibitor of Rho) were added to the cells in the presence or absence of ROSI prior to phagocytosis. Calcein-AM-labeled apoptotic J774 cells (uv-irradiated) were overlaid at a ration of 1:1 in the presence of the indicated compounds for 35 minutes. Unengulfed cells were rinsed off and the percent engulfment was scored by microscopy.

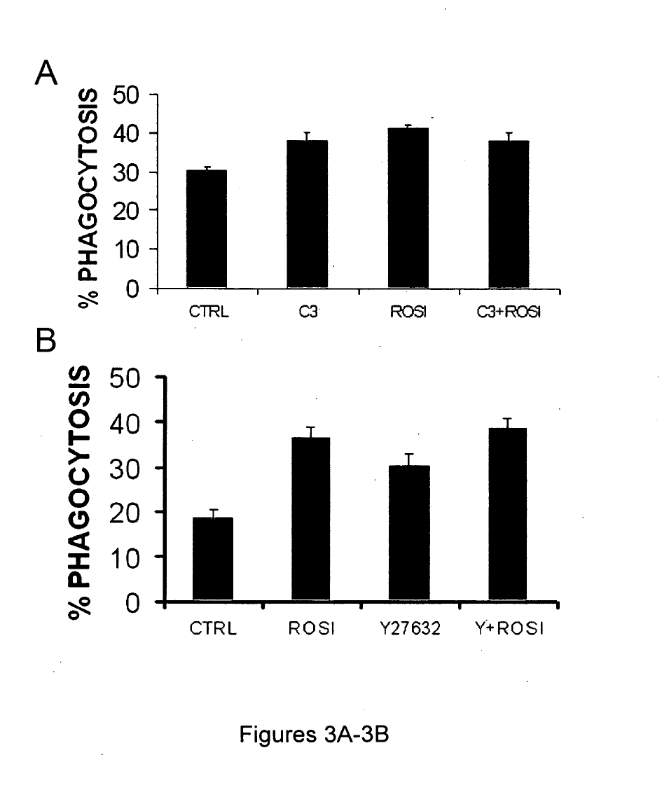

It was found that the effect of the TZD and of Rho signaling inhibitors was to enhance phagocytosis (FIG. 3A and FIG. 3B). Furthermore, phagocytosis enhancement was not additive for the TZD and the inhibitors of Rho signaling, indicating that TZDs function through the Rho pathway. Thus, TZDs and other PPAR-gamma activators, inhibitors of RhoA, and inhibitors of ROCK can be used to enhance phagocytosis. In this context, it has been shown that that treatment of macrophages with inhibitors of both RhoA and ROCK kinase can increase the clearance of apoptotic macrophages.

### Example 3

### Evaluation of the Effects of Statins on Phagocytic Clearance of Apoptotic Macrophages In Vitro

Statins are currently standard therapy for patients at risk for coronary artery disease (CAD). Therefore, in some cases, a composition useful for phagocyte enhancement therapy is administered with a statin and has effects that are additive to or synergistic with statin therapy.

In vitro studies have been performed and show that statins inhibit both RhoA, which will enhance phagocytic clearance of apoptotic cells, and Racl/Cdc42, which can inhibit this process (Muniz-Junqueira et al., Int. Immunopharmacol. 6:53, 2006; Cordle et al., J. Biol. Chem. 280:34202, 2005; Loike et al., Arterioscler. Thromb. Vasc. Biol. 24:2051-2056, 2004). When apoptotic neutrophils were used in an in vitro phagocytic uptake assay, statins showed a net enhancing effect on apoptotic cell clearance (Morimoto et al., J. Immunol. 176:7657, 2006). Accordingly, molecules that are identified as candidate phagocyte enhancer molecules can be tested for their effect on phagocyte enhancement in the presence of a statin. It is also useful to test and identify statins and derivatives thereof that have effects on phagocyte enhancement, particularly their effect on the clearance of apoptotic macrophages. Compounds that increase phagocyte enhancement in the presence of a statin are useful for combination therapy with a statin to treat coronary artery disease. Therapy with statins that are identified as having relatively weak phagocyte enhancer activity can be supplemented by combining the statin therapy with a phagocyte enhancer molecule. Statins that are identified as having high phagocyte enhancement activity are identified as being particularly useful in treatment of a subject having advanced atherosclerotic plaques. In some cases, supplementation of therapy with a phagocyte enhancer molecule is useful to achieve an even greater phagocyte enhancement effect.

Studies are conducted to further identify statins having phagocyte enhancer activity and to demonstrate the usefulness of a combination therapy using a statin and a phagocyte enhancer molecule. In these experiments, the effects of various doses and types of statins (e.g., simvastatin and atorvastatin) on phagocytic clearance of apoptotic macrophages in vitro are tested using quantification of uptake of fluorescently labeled apoptotic macrophages by phagocytic macrophages. Macrophages are rendered apoptotic by one or more methods known in the art that are relevant in vivo, e.g., FC-loading, oxidized low-density lipoprotein (oxLDL), or growth factor withdrawal.

Three conditions for macrophages are tested in these experiments; (a) untreated macrophage phagocytes; (b) phagocytes treated with inflammatory stimulators (e.g., at least one of TNFα, IL1β, IL6, CD40 ligand, or IFNγ) to mimic the milieu of advanced atherosclerotic lesions; and (c) phagocytes subjected to a number of perturbations that have been proposed to suppress phagocytosis in advanced atherosclerotic lesions, such as hypoxia and oxidative stress. Such methods are known in the art and certain methods are described herein. The system of apoptotic macrophages and phagocytic macrophages is assessed for a stimulatory or inhibitory effect of each tested statin on phagocytic clearance. Experiments are also conducted in the presence a statin with or without a phagocyte enhancer molecule. Phagocyte enhancer molecules that increase phagocyte activity in the presence of the statin are useful for treating a cardiovascular disease in conjunction with statin treatment.

Studies are also conducted to determine whether stimulatory or inhibiting effects of statins can be reproduced by farnesyl and/or geranylgeranyl transferase inhibitors, which mimic the Rho family actions of statins. The effect is also tested by examining reversal of the statin effect by low-dose mevalonate and not by cholesterol.

These studies are useful for selecting combinations of statins and phagocyte enhancers that are complementary in their activity, e.g., on enhancement of phagocytosis. In general a phagocyte enhancer that has phagocyte enhancing activity that is different than a specific statin is used in combination with the statin in combination therapy for treating or preventing cardiovascular disease.

Compounds that target mechanisms that affect other functions or activities associated with enhancing phagocytosis, such as compounds that (a) inhibit RhoA GTPase or inhibit other molecules or pathways involved in actin remodeling associated with decreased phagocytosis; or (b) that activate Rac1 or Cdc42 GTPases, or activate other molecules or pathways that promote actin remodeling associated with enhanced phagocytosis, can be identified using methods known in the art, and further tested in systems such as those described herein for their ability to function as phagocytosis enhancers. Such compounds are also useful for treating disorders that benefit from increasing phagocytosis, e.g., atherosclerosis.

### Example 4

### Rho Kinase Inhibitors

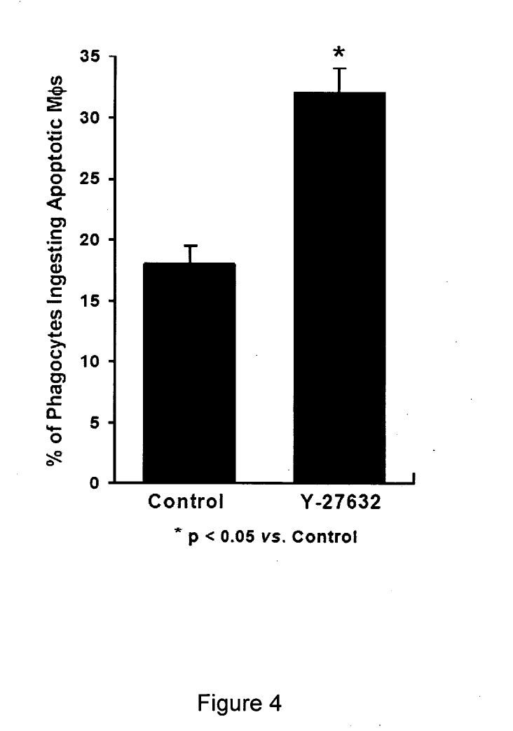

As discussed in Example 6, statins can inhibit RhoA activation. RhoA activation inhibits phagocytic clearance of apoptotic cells and so inhibitors of RhoA or the downstream RhoA effector, Rho kinase (ROCK) can enhance or at least contribute to limit or decrease Rho-mediated inhibition of phagocytic clearance. This was demonstrated in experiments in which phagocytic uptake of apoptotic macrophages was assayed using the ROCK inhibitor Y-27632 (trans-4-[(1R)-1-aminoethyl]-N-pyridin-4-ylcyclohexanecarboxamide). In these experiments, peritoneal macrophages (phagocytes) were treated for one hour in the presence or absence of 10 μM Y-27632. Calcein-AM-labeled (green) apoptotic J774 cells (UV-irradiated) were then added to the phagocytes at a ratio of 1:1, in the absence or presence of Y-27632. After 45 minutes, non-internalized cells were removed by rinsing, and the percentage of phagocytes that had internalized labeled apoptotic macrophages was quantified by fluorescence microscopy. Results are depicted in FIG. 4 as the as the mean ±SEM; n=3 fields of cells, each containing approximately 150 cells. In these experiments, those phagocyte samples treated with Y-27632 demonstrated an increase in the percentage of phagocytes ingesting apoptotic macrophages.

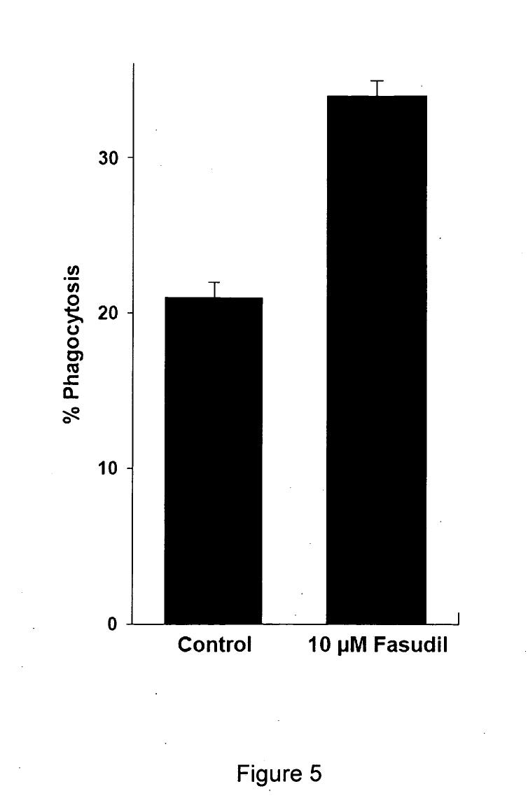

In a similar experiment, J744 murine macrophages (phagocytes) were pretreated in the presence of absence of the ROCK inhibitor fasudil (10 μM) for one hour. The phagocytes were then incubated for 45 minutes, with or without fasudil, with fluorescently labeled UV-induced apoptotic J774 macrophages (“UV-Ams”). The percentage of phagocytes that had engulfed at least one UV-AM was quantified using fluorescent microscopy. The percentage phagocytosis was increased in those samples treated with fasudil (FIG. 5), further demonstrating the efficacy of inhibitors of the RhoA pathway (e.g., ROCK inhibitors) for increasing phagocytosis of apoptotic macrophages. Such compounds are useful for treating cardiovascular disease.

Other compounds that may be useful as enhancers of phagocytic clearance can be tested in this system. This system can also be used to identify compounds that are useful in combination with statins, e.g., by treating cells with statins and testing the statin-treated cells in the presence and absence of a candidate phagocyte enhancer compound. A candidate phagocyte enhancer compound that increases phagocyte clearance of apoptotic macrophages can be useful for treating cardiovascular disease in combination with a statin.

This Example illustrates a method of identifying compounds that are useful for enhancing phagocytic clearance. An example of such an additive compound includes, without limitation, fasudil.

In other methods useful for identifying compounds that enhance phagocytosis, compounds known to promote actin signaling and remodeling that are associated with phagocytosis are tested for their ability to act an phagocyte enhancers to promote clearance of apoptotic macrophages using methods such as those described herein. Actin activities that are related to promoting phagocytosis and thus are targets for promoting phagocytosis or that can be assayed in evaluations of phagocytosis enhancers (i.e., such activity is increased in the presence of a certain phagocytosis enhancers) are known in the art (for example, May et al., 2001, J. Cell Sci. 114(6):1061-1077). Compounds that promote activities associated with promoting actin signaling and remodeling are known in the art, or can be identified using methods that identify such compounds. Examples of such compounds include, without limitation, AtSCAR1 and ZmSCAR1 (Egile et al., 2004, Proc. Natl. Acad. Sci. USA 2004 Nov. 16; 101(46):16379-84). Such compounds are candidate phagocytosis enhancers that are useful for enhancing phagocytic clearance of apoptotic cells.

### Example 5

### Evaluation of the Effects of Statins and Fasudil on Phagocytic Clearance of Apoptotic Macrophages In Vivo

Compounds can be tested for their ability to enhance phagocyte clearance of apoptotic macrophages in the presence of a statin in vivo. For example, in vivo studies are conducted using four groups of mice; mice receiving no treatment, mice treated with statin alone, mice treated with ROCK inhibitor alone, and mice treated with statin plus ROCK inhibitor. Chow-fed Apoe^{-/-} mice are used in these experiments because, in contrast to the profound lowering of LDL by statins in Western diet-fed Ldlr-1 mice (Wang, et al., Atherosclerosis 162:23, 2002), statins have only modest effects on plasma cholesterol, The drugs are administered only after early-mid lesions are established in the mice to focus on advanced lesional events and to mimic a common treatment scenario with humans. Thus, 15 week old Apoe−/− mice are administered the drugs (statin, ROCK inhibitor, or both) for 10 weeks. Plasma from the mice is assayed for total cholesterol, HDL-cholesterol, and triglycerides. The atherosclerosis endpoints are the indices of plaque vulnerability (e.g., necrosis, apoptosis, inflammation, and fibrous cap thickness) and advanced lesional phagocytic efficiency that is assayed using methods known in the art, e.g., as described herein. These experiments demonstrate the effect of a statin on phagocytic clearance of apoptotic macrophages.

This in vivo system is also useful for identifying phagocyte enhancer compounds that are compatible for use with a statin. To perform such an identification, mice are treated with a selected statin or the selected statin with a test compound. A test compound that increases phagocytic clearance of apoptotic macrophages or improves one or more features associated with such activity is useful as a phagocyte enhancer, e.g., in combination with a statin.

Experiments are performed to determine whether apoptotic macrophages injected i.p. into drug-treated versus control mice are more efficiently cleared. These assays are performed using the methodology of Mitchell et al., J. Am. Soc. Nephrol. 13:2497, 2002.

In these experiments, a statin (e.g., simvastatin or atorvastatin) and a ROCK inhibitor (i.e., fasudil or Y-27632) are selected. In a study by Wang et al, simvastatin was added to the chow at a concentration of 0.15%, and in an Apoe−/− atherosclerosis study by Grothusen et al. (Atherosclerosis 182:57, 2005), atorvastatin was added to the drinking water at a dose of 1 mg/kg body weight. These dosages are provided as guidance and other dosages can be used. Fasudil is generally used as the ROCK inhibitor because it can be administered to mice in the drinking water (Wang et al., Circulation 111:2219, 2005). In contrast, Y-27632 is given via daily i.p. injections (Mallat et al., Circ. Res. 93:884, 2003). In the case of fasudil, the drug is added to an animal's drinking water at a concentration of 1 mg/ml, as described in Wang et al. Such mice are also treated in the presence and absence of a compound that is being tested as a phagocyte enhancer. Compounds that increase phagocyte clearance of apoptotic macrophages or increase features indicative of such activity, e.g., in the presence of a statin, are useful for combination therapies with a statin for treating cardiovascular disease.

### Example 6

### Materials and Methods

The Materials and Methods in this Example are illustrative of materials and methods that can be used for certain assays described herein. They are specifically used for the experiments of Examples 7-13, infra.

Falcon tissue culture plasticware was purchased from Fisher Scientific Co. Cell culture media, reagents and heat-inactivated FBS (GIBCO BRL) were from Invitrogen. Alexa Fluor 488 annexin V, Alexa Fluor 594 annexin V, Calcium Green™-acetoxymethyl ester (AM) were obtained from Molecular Probes, Inc. [³H]Cholesterol and [¹⁴C]oleate were purchased from Perkin-Elmer Life Sciences, Inc. All other chemicals and reagents were from Sigma, and HPLC grade organic solvents were from Fisher Scientific Co. Low-density lipoprotein (LDL; d 1.020-1.063 g/ml) was isolated from fresh human plasma by ultracentrifugation (Havel et al., 1955, J. Clin. Invest. 34:1345-1353). Acetyl-LDL was prepared by reaction of LDL with acetic anhydride as described in Basu et al. (1976, Proc. Natl. Acad. Sci. USA 73:3178-3182). Compound 58035 (3-[decyldimethylsilyl]-N-[2-(4-methylphenyl)-1-phenylethyl]propanamide), an inhibitor of acyl-CoA:cholesterol O-acyltransferase (ACAT), was from Dr. John Heider, formerly of Sandoz, Inc. (Ross et al., 1984, J. Biol. Chem. 259:815-819). PS1145 was obtained from Millennium Pharmaceuticals (Hideshima et al., 2002, J. Biol. Chem. 277:16639-16647). LY294002 was purchased from MC Biosciences. Anti-phospho-AKT antibody was obtained from Cell Signaling Technology, and monoclonal anti-β-actin antibody was from Santa Cruz Biotechnologies, Inc. HRP-conjugated donkey anti-mouse and donkey anti-rabbit IgG secondary antibodies were purchased from Jackson ImmunoResearch Laboratories.

Peritoneal Macrophages

For routine experiments, peritoneal macrophages were collected from 8-10 week old female C57BL6J mice that had been injected intraperitoneally with concanavalin A or with methyl-BSA after immunization with this compound, as described previously (Li et al., 2006, J. Biol. Chem. 281:6707-6717; Cook, et al., 2003, J. Immunol. 171:4816-4823). Cells were cultured in medium containing Dulbecco's modified Eagle's medium (DMEM), 10% FBS, 100 units/ml penicillin/streptomycin, and 20% L-cell-conditioned medium for at least 48 hours. The medium was replaced every 24 hours until the macrophages were confluent. For some experiments, as indicated, peritoneal macrophages were obtained from Acat1−/− (Soat1−/−) mice on the C57BL6/J background (Accad et al., 2000, J. Clin. Invest. 105:711-719). Some experiments also used peritoneal macrophages from Bcl2flox and Bcl2flox×LysMCre mice, also on the C57BL6 background. The Bcl2^{flox} mice were made using a 12.5-kb mouse genomic DNA fragment obtained from a murine 129 lambda genomic library. This genomic fragment contained exon 2 of the Bcl2 gene. A 3.5-kb EcoRI-XbaI fragment was cloned to serve as short arm and middle arm for the final construct. A loxP site along with a new EcoRI site was inserted into the NcoI site of this fragment, and it was then inserted at the 3′ end of a Neo cassette flanked by two loxP sites. The long arm was a 6-kb BglII-BglII fragment, which was inserted at 5′ of the floxed Neo cassette. Ten micrograms of this targeting vector was linearized by AscI and then transfected by electroporation into 129 embryonic stem cells, which were then used to generate the Bcl2flox mice. LysMCre mice (Clausen et al., 1999, Transgen. Res. 8:265-277) were crossed into the C57BL6 background and used as described in Zhang et al. (2000, J. Biol. Chem. 275:35368-35376).

Generation of Free Cholesterol Induced Apoptotic Macrophages (FC-AMs)

Macrophages cultured as described above were incubated for 16-20 hours with medium containing 100 μg/ml of acetyl-LDL and 10 μg/ml of the ACAT inhibitor 58035 to induce early apoptosis (“FC-AMs”). In some experiments, Acat1^{-/-} macrophages were used instead of the ACAT inhibitor. Typically, 30-40% of macrophages were apoptotic and less than 5% were late apoptotic or necrotic as assessed by annexin V and propidium iodide staining, respectively.

Phagocytosis

FC-AMs were removed from the culture dish and cultured for 30 minutes with a monolayer of fresh macrophages (“phagocytes”) at an approximate ratio of 1:5 (FC-AMs:phagocytes). In certain experiments, the FC-AMs were labeled with Alexa Fluor 488 annexin V or Calcium Green™-AM for 20 minutes prior to addition to the phagocytes in order to mark those phagocytes that had ingested the FC-AMs (“ingesting phagocytes,” or “IPs”). The non-ingested apoptotic cells were then removed by thorough rinsing as described in Li et al. (2006, J. Biol. Chem. 281:6707-6717), and the phagocytes were incubated in fresh medium for the indicated times. In some experiments, the phagocytes were incubated during the post-ingestion incubation in medium containing acetyl-LDL and 58035 to maintain FC levels in the (ingesting phagocytes) IPs, inhibitors of Akt or NFκB, or various combinations of these reagents. To assay apoptosis in the phagocytes, the cells were stained with Alexa Fluor 594 annexin V and viewed by fluorescence microscopy. For quantification, 4-6 representative fields of cells at 40× magnification were counted to determine the number of apoptotic phagocytes and total phagocytes for each condition.

Whole-Cell Cholesterol Esterification Assay in Phagocytes

After a 30 minute incubation of phagocytes with FC-AMs that were made using acetyl-LDL and macrophages from Acat1^{-/-} mice (i.e., no ACAT inhibitor), non-ingested apoptotic cells were removed, and the phagocytes were incubated in fresh medium containing [¹⁴C]oleate for specific times. The cells were then washed twice with phosphate-buffered saline, air-dried, and then extracted twice with 500 μl of hexane/isopropyl alcohol (3:2, v/v) for 30 minutes at room temperature. Cholesterol esterification activity was then determined in lipid extracts of the cells by measuring the cellular content of cholesteryl [¹⁴C]oleate by thin-layer chromatography (Tabas et al., 1987, J. Clin. Invest. 79:418-426). The lipid-extracted cells were dissolved in 1 ml of 0.1 N NaOH and assayed for protein by the method of Lowry.

[³H]Cholesterol Efflux Assay

[³H]cholesterol-labeled FC-AMs were prepared using acetyl-LDL that had been labeled with [³H]cholesterol. Specifically, 1 mg acetyl-LDL was incubated with 10 μCi [³H]-cholesterol for 30 minutes at 37° C. and then added to a 100-mm dish of macrophages in 10 ml medium containing 10 μg/ml 58035. After 18-20 hours of incubation to induce apoptosis, the monolayer was rinsed thoroughly with PBS. The labeled FC-AMs were then added to a fresh monolayer of phagocytes for 30 minutes. The non-ingested apoptotic cells were removed by intensive washing, and the phagocytes were further incubated in fresh medium for the indicated times. An aliquot of medium was collected at the indicated time points, and the radioactivity was quantified by liquid scintillation counting. The cells were dissolved in 1 ml of 0.1 N NaOH at room temperature for 5 hours, and the radioactivity in the cell lysates was quantified. Cholesterol efflux was calculated as [(media cpm)÷(cell+media cpm)]×100.

Cellular Free Cholesterol Mass Assay in Phagocytes

Phagocytes were washed two times with cold PBS and then extracted twice with 0.5 ml of hexane/isopropyl alcohol (3:2, v/v) for 30 minutes at room temperature. In certain experiments, the FC-AMs were labeled with Alexa Fluor 488 annexin V before exposure to phagocytes, and then the phagocytes were subjected to FACS sorting to separate IPs (green) and non-IP macrophages (non-green). The free cholesterol mass was determined by gas-liquid chromatography as described previously (Shiratori et al., 1994, J. Biol. Chem. 269:11337-11348). The cell monolayers were dissolved in 1 ml of 0.1 NNaOH, and aliquots were assayed for protein by the method of Lowry et al. (1951, J. Biol. Chem. 193:265-275).

Western-Blot Analysis

Whole-cell lysates were prepared by homogenizing cells with Laemmli sample buffer from BioRad, as described previously (Li et al., 2006, J. Biol. Chem. 281:6707-6717). These lysates were fractioned on 4-20% gradient SDS-polyacrylamide gels (Invitrogen) and then transferred to nitrocellulose membranes. After blocking the membranes with 5% (w/v) nonfat milk in Tris-buffered saline, 0.1% Tween-20 (TBST) at room temperature for 1 hour, they were incubated overnight at 4° C. with primary antibody. The membranes were then incubated with HRP-conjugated secondary antibody, and the immunoreactive protein bands were detected by ECL chemiluminescence (Pierce).

Statistics

Data are presented as mean ±S.E.M. of triplicate experiments unless stated otherwise. Absent error bars in the bar graphs signify S.E.M. values smaller than the graphic symbols.

### Example 7

### Ingestion of FC-AMs does not Induce Apoptosis in ACAT-Inhibited Phagocytes

Advanced lesional macrophages are putatively dysfunctional with respect to ACAT activity. A previously described experimental system in which FC-induced apoptotic macrophages (FC-AMs), a model of advanced lesional macrophage death, were added briefly to a fresh monolayer of untreated macrophages (phagocytes) to allow internalization (Li et al., 2006, J. Biol. Chem. 281:6707-6717). FC-AMs were created by incubating macrophages for 18 hours with acetyl-LDL, a commonly used model of an atherogenic lipoprotein, plus an inhibitor of ACAT-mediated cholesterol esterification, which is designed to mimic the putative dysfunction of ACAT in advanced lesional macrophages (Tabas et al., 2002, J. Clin. Invest. 110:905-911). Thirty minutes after FC-AM addition, the phagocytes were rinsed thoroughly to remove non-ingested apoptotic cells, and then the phagocytes incubated in fresh serum-containing medium for various periods of time. To detect the subpopulation of phagocytes that actually ingested the FC-AMs, the apoptotic cells were labeled with the green vital fluorescent dye Calcium Green™-AM prior to adding them to the phagocytes. The subpopulation of phagocytes that ingest Calcium Green™-AM-labeled FC-AMs are referred to as “ingesting phagocytes,” or “IPs.” Previous studies documented that the labeled IPs represent phagocytes that have fully ingested FC-AMs.

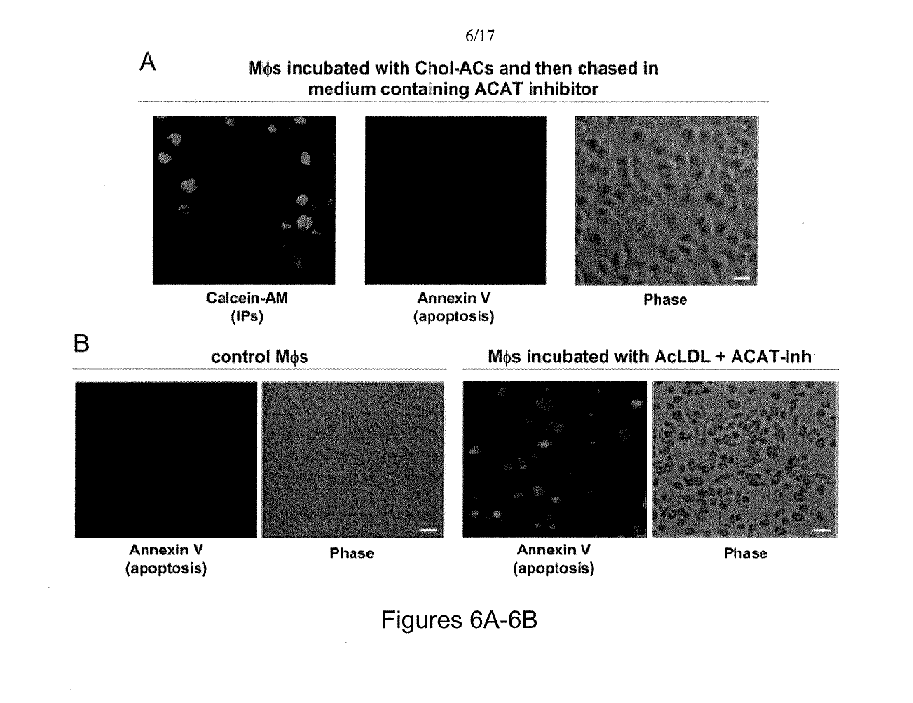

The first question addressed was whether the ingestion of FC-AMs by ACAT-inhibited phagocytes would induce phagocyte death via FC toxicity or by other possible mechanisms. Initial observation of the phagocytes by phase microscopy showed no signs of cytotoxicity even 24 hours after FC-AM ingestion. To look for more subtle signs of cytotoxicity, the phagocytes were labeled with Alexa Fluor 594-conjugated annexin V (red) to detect externalized phosphatidylserine, a sign of early-mid-stage apoptosis. As shown in FIG. 6A, a subpopulation of phagocytes were labeled, indicating uptake of the Calcium Green™-AM-labeled FC-AMs. Remarkably, although consistent with the phase microscopy observations, these ACAT-inhibited IPs were not labeled by annexin V (FIG. 6A, middle panel). As a positive control for annexin staining, macrophages that were loaded directly with FC by incubation with acetyl-LDL plus an ACAT inhibitor stained intensely with annexin V, as expected (FIG. 6B). Thus, ACAT-inhibited phagocytes that have ingested FC-AMs, a very rich source of cholesterol, do not undergo apoptosis.

### Example 8

### Neither a Cholesterol-to-ER Trafficking Defect Nor the Lack of Engagement of the Type A Scavenger Receptor can Explain the Lack of FC-AM-Induced Apoptosis in Ingesting Phagocytes

FC-induced macrophage apoptosis is dependent on FC trafficking to the endoplasmic reticulum (ER), which triggers the ER-based stress pathway known as the unfolded protein response (UPR). Therefore, one possible mechanism for the lack of apoptosis in IPs is that FC-AM-derived cholesterol cannot traffic to the ER. This might occur, for example, if the cholesterol were trapped in phagolysosomes. To evaluate this possibility, advantage was taken of the fact that cholesterol trafficking to the ER results in cholesterol esterification by the ER-specific enzyme ACAT. Thus, as a marker of cholesterol trafficking to the ER, it was determined whether FC-AMs were able to stimulate cholesterol esterification in macrophage phagocytes. A standard live-cell assay for cholesterol esterification was used in which macrophages are incubated with [¹⁴C]oleate in the absence or presence of a source of cholesterol and then assayed for cholesteryl [¹⁴C]oleate formation.

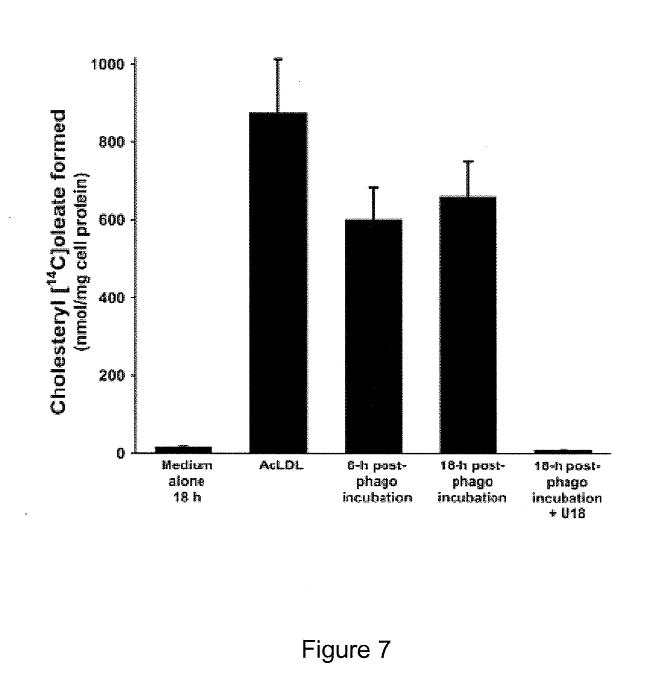

Exposure of the phagocytes to FC-AMs resulted in a marked increase in cholesterol esterification (FIG. 7). Moreover, this increase was completely blocked by compound U18666A, which blocks cholesterol trafficking from degradative organelles to peripheral sites, including the ER. These data indicate that cholesterol-derived from the ingestion of FC-AMs can, in fact, traffic to the ER. Consistent with these data, the unfolded protein response (UPR) effector CHOP was induced in the phagocytes within a few hours after ingestion of FC-AMs. Therefore, the explanation for the lack of apoptosis in IPs must lie elsewhere.

FC-induced apoptosis in macrophages requires UPR activation in combination with engagement of the type A scavenger receptor (SRA), both of which occur with acetyl-LDL-induced FC loading. Consistent with this model, apoptosis can be triggered by adding separate “hits” in this pathway, namely a non-SRA UPR activator (e.g., thapsigargin) plus a non-UPR SRA ligand (e.g. fucoidan), but not by adding either reagent alone. Moreover, macrophages with decreased or absent SRA are much less susceptible to FC-induced apoptosis (DeVries-Seimon et al., 2005, J. Cell Biol. 171:61-73). Therefore, it is possible that lack of engagement of the SRA by FC-AMs or a decreased SRA levels in IPs accounts for the lack of FC-AM-induced apoptosis. To test these possibilities, phagocytes that had ingested FC-AMs were incubated with the SRA ligand fucoidan. However, fucoidan did not induce apoptosis in the IPs. In addition, immunoblot experiments showed that SRA protein levels in IPs were not lower than those in control macrophages. Therefore, lack of SRA engagement or receptors cannot explain the resistance to apoptosis in IPs.

### Example 9

### Marked Cholesterol Efflux from IPs Post-Ingestion of FC-AMs

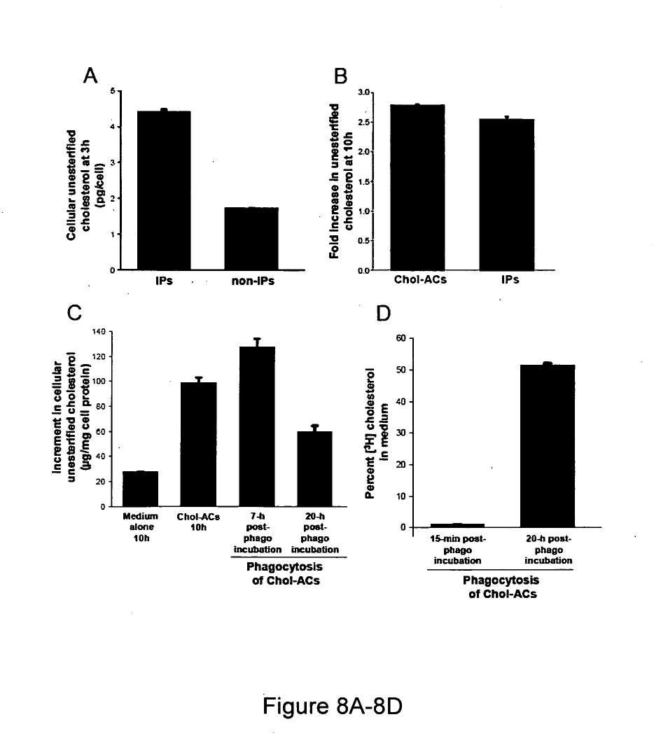

Despite the prediction that ACAT-compromised phagocytes ingesting FC-AMs should acquire large amounts of FC, it was possible that something might limit FC accumulation over time. In particular, it was possible that while large amounts of cholesterol almost certainly enter the cells initially, the cholesterol may get effluxed before apoptosis was triggered. To test this possibility, ACAT-inhibited macrophage phagocytes were incubated with FC-AMs labeled with fluorescent annexin V to distinguish IPs from non-IPs. After a 3 hour post-ingestion incubation, the IPs and non-JPs were separated by FACS and assayed for cholesterol mass by gas-liquid chromatography. As expected, the IPs accumulated a substantial amount of FC compared to non-JPs (FIG. 8A). Next the fold increase in FC accumulation in ACAT-inhibited IPs was directly compare with the fold increase in FC-AMs, because the latter represents a level known to induce apoptosis. As shown in FIG. 8B, the fold increase in FC accumulation at 10 hours was similar under each condition. In addition, the absolute level of intracellular FC in 7 hour IPs was even greater than that in 10 hour FC-AMs (FIG. 8C, second and third bars). Thus, the initial amount of FC accumulating in the IPs should be adequate to induce apoptosis. However, as shown in FIG. 8C (fourth bar), intracellular FC in IPs drops substantially by 20 hours post-ingestion. Moreover, there was marked efflux of ingested cholesterol during the 20 hour post-ingestion period (FIG. 8D). These data raised the possibility that ACAT-inhibited IPs were protected from FC-AM-induced apoptosis, at least in part, by efflux of FC before irreversible death signaling occurred. This idea is supported by the finding that while macrophages loaded with FC for a continuous 18-20 hour period undergo apoptosis (FIG. 1B and DeVries-Seimon et al., 2005, J. Cell Biol. 171:61-73), macrophages loaded with FC for 8-10 hours and then chased in control medium for 10 hours, which mimics the decrease of FC levels that naturally occurs in IPs, survive.

### Example 10

### IPs are Partially Resistant to Apoptosis Even when Intracellular Fc Levels are Maintained at a High Level

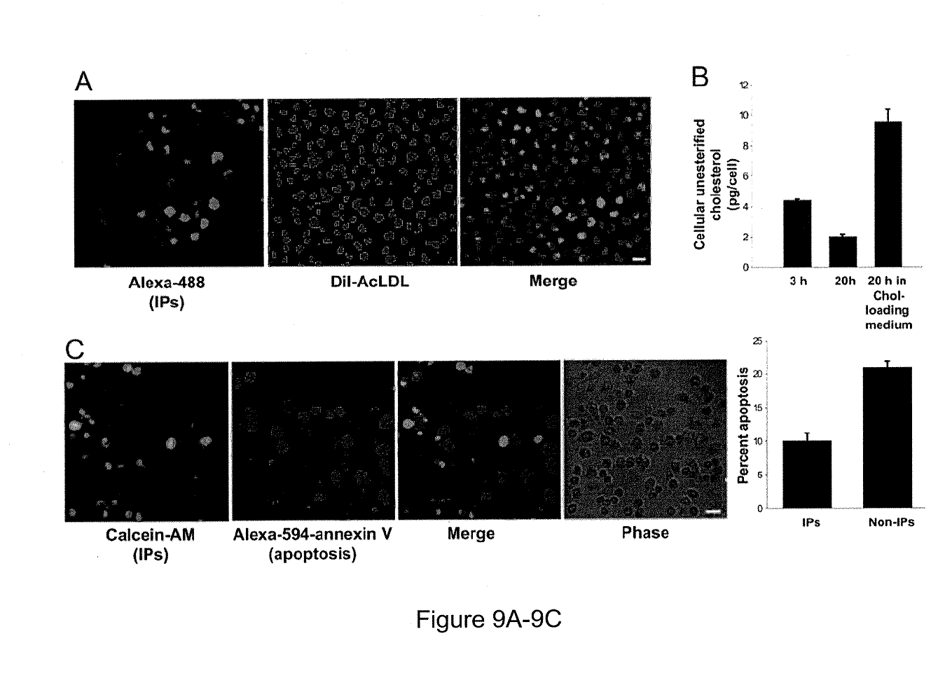

If the efflux of intracellular cholesterol were the sole mechanism of survival in ACAT-inhibited IPs, then it should be possible to induce apoptosis by maintaining their FC levels over the course of the 20 hour post-ingestion period. To maintain the FC levels in IPs, IPs were incubated with acetyl-LDL plus ACAT inhibitor during the 20 hour post-ingestion chase period. FIG. 9A shows that IPs were able to internalize acetyl-LDL, and, as expected, the FC levels in these cells were maintained for 20 hours at a 4-5-fold higher level of FC than when FC loading was not conducted during the 20 hour period (FIG. 9B). To determine the susceptibility to apoptosis of IPs treated under these persistently high-FC conditions, phagocytes were incubated with Calcium Green™-AM-labeled FC-AMs (green) to distinguish IPs from non-IPs. After a 20 hour post-ingestion period under FC-loading conditions, the phagocytes were stained with fluorescent annexin V (red) to detect apoptosis. Although some of these FC-loaded IPs became apoptotic, apoptosis was approximately two-fold more prevalent in non-JPs (red only) than in IPs (red and green) (FIG. 9C). Thus, the process of phagocytosis of FC-AMs appears to partially protect the phagocytes from apoptosis even when intracellular FC levels are maintained at a very high level.

### Example 11

### NFκB and PI-3 kinase/AKT signaling pathways are required for the survival response of persistently FC-loaded IPs

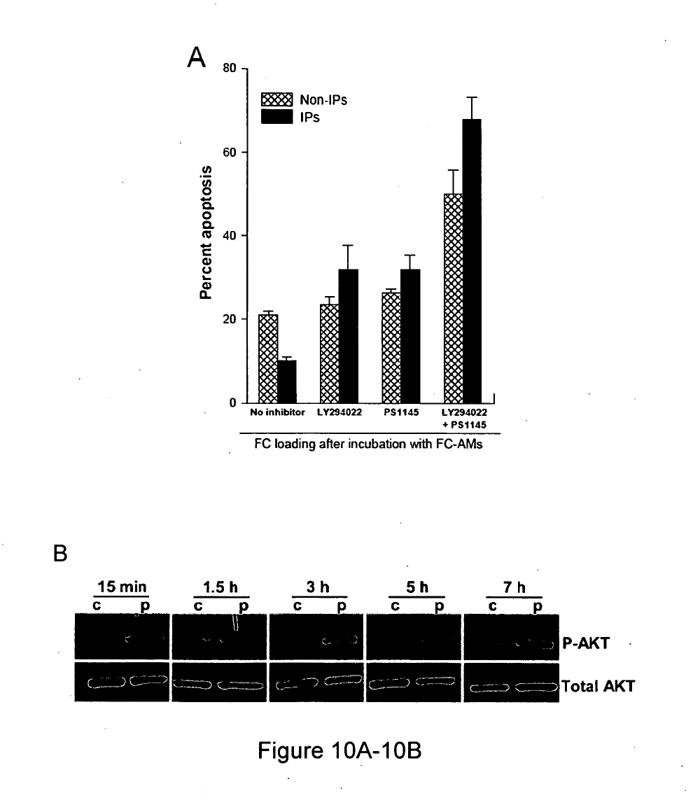

A recent study demonstrated that exposure to FC-AMs activates NFκB signaling in IPs (Li et al., 2006, J. Biol. Chem. 281:6707-6717). Because NFκB is known to signal survival responses in cells, the functional significance of NFκB signaling in the survival response of IPs against FC-induced apoptosis was investigated. Compound PS1145, a specific inhibitor of IKK that efficiently inhibits NFκB signaling in IPs, was added to IPs during the 20 hour post-ingestion/FC-loading period. As demonstrated previously, IPs are partially resistant to FC-induced apoptosis compared to non-JPs (FIG. 10A, first pair of bars). In the setting of PI-3 kinase/Akt inhibition, FC-induced apoptosis in non-JPs was only slightly increased, while that in IPs was markedly increased (FIG. 5A, second pair of bars). A very similar effect was seen when IKK was inhibited (FIG. 5A, third pair of bars). The NFkB and Akt survival pathways might represent independent, complementary survival pathways or they may signal through a common final mediator. The former idea is more likely, because Akt is activated at relatively early time points post-phagocytosis (FIG. 10B), while NFκB is activated at later time points (i.e., >6 hours after phagocytosis (Li et al., 2006, J. Biol. Chem. 281:6707-6717)). To investigate this point, the effect of the combination of both inhibitors was compared to the effect of each inhibitor alone. Although apoptosis in both IPs and non-IPs was increased by combined inhibitor treatment (FIG. 10A, fourth pair of bars), the fold-increase in IP apoptosis (6.8) was markedly greater than that in non-IP apoptosis (2.4) when compared to the no-inhibitor control. Moreover, apoptosis in IPs treated with both inhibitors (68.0±5.4%) was approximately additive to that seen with each inhibitor alone (32.0±5.7% and 32.0±3.4%, respectively). Thus, NFκB and PI-3 kinase/Akt, through complementary pathways, play critical roles in the ability of IPs to remain viable despite high levels of FC-loading.

### Example 12

### Bcl-2 is Involved in the Survival Response of Persistently FC-Loaded IPs

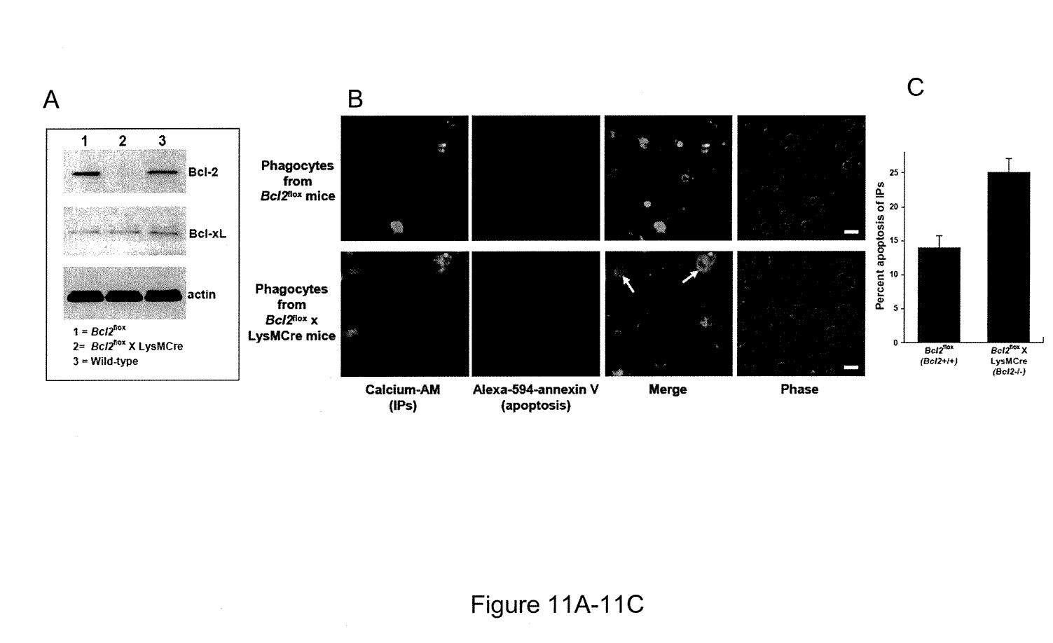

Bcl-2 is a downstream anti-apoptotic protein that can help mediate the survival pathways induced by NFκB and/or Akt. Moreover, Bcl-2 levels were found to be transiently increased in phagocytes after exposure to FC-AMs. Therefore, the possibility was considered that Bcl-2 played a role in the partial survival response of FC-loaded IPs. To test this possibility, peritoneal macrophages were used that were from mice with macrophage-targeted Bcl-2 deficiency (Bcl2^{flox}×LysMCre) and from littermate control mice (Bcl2^{flox}) (Clausen et al., 1999, Transgen. Res. 8:265-277)). As expected, the macrophages from the experimental mice express no detectable Bcl-2 while those from the littermate control mice express normal levels of Bcl-2 (FIG. 11A). Control and Bcl-2-deficient macrophages were used as the source of phagocytes to determine whether the absence of Bc1-2 might decrease the survival response in FC-loaded IPs. As shown in FIG. 11B, top row of images, the Bcl-2-control IPs showed a relatively low level of FC-induced apoptosis, as expected from the previous data. In contrast, substantially more apoptotic IPs were seen when Bcl-2-deficient macrophages were used as phagocytes (FIG. 11B, bottom row of images). The quantified data are shown in FIG. 11C. These data indicate that Bcl-2 plays a partial role in the survival response of FC-loaded IPs.

These data indicate that compounds that increase or stabilize Bcl-2 expression or activity can be used to increase survival of FC-loaded IPs, and thus are also useful for treating or preventing atherosclerosis.

### Example 13

### IPs are Partially Resistant to UV-Induced Apoptosis Through a Mechanism that Relies Primarily on Akt Signaling

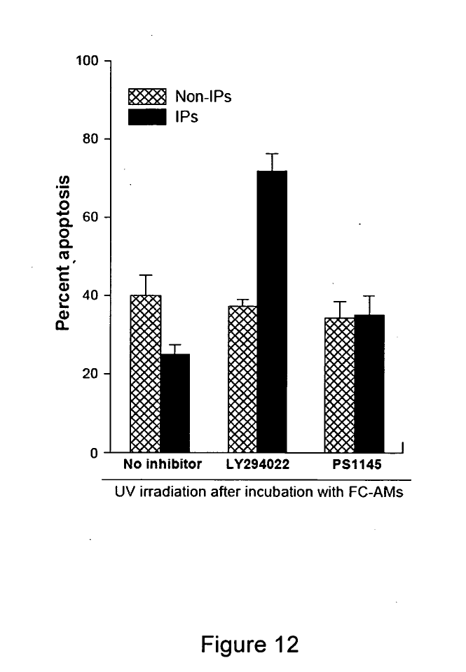

To determine whether the partial resistance of IPs to subsequent apoptotic stimuli might extend beyond FC loading, post-ingestion IPs were exposed to a dose of UV irradiation that is known to induce apoptosis in macrophages (Li et al., 2006, J. Biol. Chem. 281:6707-6717). As shown in FIG. 12, first pair of bars, IPs were partially resistant to UV-induced apoptosis. Inhibition of PI-3kinase/Akt signaling caused a marked increase in apoptosis in IPs but not in non-IPs (FIG. 12, second pair of bars). In contrast, inhibition of NFκB was associated with only a small increase in IP apoptosis compared to the no-inhibitor control, and there was no effect when compared to non-IPs (FIG. 12, third pair of bars). Thus the ability of IPs to partially survive a death insult extends beyond FC-induced apoptosis, although the relative importance of specific survival pathways appears to differ depending upon the nature of the insult.

The data of Examples 7-13 illustrate an experimental model that contains several key features of advanced atherosclerotic lesions. This model can be used to identify compounds that affect phagocytosis, e.g., compounds that enhance phagocytosis or functions associated with advanced phagocytosis. Compounds identified in the model can be further tested to confirm their efficacy, e.g., for reducing atherosclerotic lesions such as advanced atherosclerotic lesions. The studies also reveal that phagocytic macrophages rely on (e.g., activate) several layers of protective mechanisms that result in their prolonged survival. Accordingly, compounds that enhance activity of the NFκB pathway, enhance activity of the Akt pathway or both can function as phagocyte enhancers, and are useful for treating disorders that benefit from enhancement of phagocytic activity such as atherosclerosis, because they increase the survival of phagocytes ingesting FC-AMs. For the same reason, compounds that promote cholesterol efflux can be useful.

The data provided herein demonstrate that phagocytes that ingest cholesterol-loaded apoptotic macrophages call into play a number of survival mechanisms that keep the phagocyte alive and healthy despite the fact that the phagocytes are ingesting very high levels of cholesterol. These findings demonstrate phagocytes have the capacity to be treated to enhance their uptake of apoptotic cells without damaging the phagocytes themselves, thus providing a useful treatment method for, e.g., cardiovascular disease such as atherosclerosis.

### Example 14

### Role of MerTK in Phagocytic Clearance of Apoptotic Bodies

It has been shown that the macrophage MerTK receptor is important for the ingestion of FC-induced apoptotic macrophages, whereas a number of other phagocytic receptors were shown not to be involved (Li et al., J. Biol. Chem. 281:6707, 2006). In addition, it has been shown that the MerTK receptor partially suppresses the pro-inflammatory response by phagocytes that have been exposed to the apoptotic macrophages. The following experiment is conducted to demonstrate that the MerTK receptor functions similarly in atherosclerotic lesions. These experiments are conducted using Mer^{kd} mice on the Apoe^{-/-} background. Mer^{kd} mice have a MerTK mutation that inactivates the MerTK receptor (Matsushima et al., 1999, J. Immunol. 162: 3498-3502.

In these experiments, Mer^{kd} mice on the C57 background are bred with Apoe^{-/-} mice to obtain 20-30 male and female mice that are Mer^{+/+}, Apoe^{-/-} and Mer^{kd}, Apoe^{-/-}. Mer^{kd} mice have no reported developmental abnormalities, demonstrate normal growth, and do not have global defects in immunity. They are more susceptible to endotoxic shock, but survive normally under control conditions. The mice are maintained on chow diet and analyzed at 10 weeks (early atherosclerosis) and 20 weeks (advanced atherosclerosis). Plasma from the mice is assayed for total cholesterol, HDL-cholesterol, and triglycerides. Aortic roots and brachiocephalic arteries (BCA) from the mice are analyzed for total lesion and necrotic area, macrophage apoptosis (using TUNEL staining), and fibrous cap thinning or rupture. Inflammation is assessed by analysis of lesional T cell numbers and inflammatory cytokine mRNA by laser capture microdissection-QT-PCT. In addition, the lesions are subjected to the analysis of Schrijvers et al. (Arterioscler. Thromb. Vasc. Biol., 2005, 25: 1256-1261) for the appearance of apoptotic bodies appearing inside vs. outside phagocytic macrophages.

Mer deficiency will lead to more extracellular apoptotic bodies in advanced lesions, indicative of a defect in phagocytic clearance and late lesional necrosis is accelerated because there is more post-apoptotic necrosis (i.e., due to lack of clearance of the apoptotic macrophages) and increased inflammation compared to controls that are not deficient to Mer.

### Example 15

### Mutation of the MerTK Receptor Promotes Apoptotic Cell Accumulation and Plaque Necrosis in Advance Atherosclerotic Lesions of Apolipoprotein E-Deficient Mice

Vulnerable atherosclerotic plaques are characterized by large necrotic cores, which result from macrophage apoptosis coupled with defective phagocytic clearance (efferocytosis) of apoptotic cells. It has been shown that macrophages with a tyrosine kinase-deficient MerTK receptor (Mertk^{KD}) have a defect in phagocytic clearance of apoptotic macrophages in vitro. In these experiments, the effect of the Mertk^{KD} mutation on apoptosis and plaque necrosis in advanced atherosclerosis is examined.

Mertk^{KD};Apoe−/− mice and control Apoe−/− mice were fed a Western-type diet for 10 or 16 wks, and aortic root lesions were analyzed for apoptosis and plaque necrosis. Plaques of the Mertk^{KD};Apoe−/− mice had a ˜70% increase in TUNEL-positive apoptotic cells (p<0.05). The necrotic areas of the more advanced (16-wk) Mertk^{KD}; Apoe−/− plaques were ˜75% greater than those in the plaques of 16-wk-fed Apoe−/− mice (p<0.05). These findings occurred despite similar lipoprotein profiles and similar overall lesion area in the two groups of mice.

Prevention and regression of human atherothrombotic vascular disease requires an understanding of the underlying mechanisms that can promote advanced plaque progression. Acute coronary artery syndromes can be associated with the presence of so-called vulnerable plaques. These plaques can be characterized by focal thinning of the fibrous cap, a high level of inflammatory cytokines and matrix proteases, apoptosis of intimal cells, and expansion of a lipid-laden necrotic core (J Interv Cardiol. 2002; 15:439-46). Expansion of the latter can be the consequence of accelerated macrophage apoptosis coupled with defective phagocytic clearance (efferocytosis) (Arterioscler Thromb Vasc Biol. 2005; 25:2255-64; Cardiovasc Res. 2007; 73:470-80). Defective phagocytic clearance can lead to post-apoptotic cellular necrosis and release of proinflammatory and pro-thrombotic intracellular debris (Chest. 2006; 129:1673-82). Previous reports have provided in-vivo evidence that efferocytosis is impaired in advanced atherosclerosis (Arterioscler Thromb Vasc Biol. 2005; 25:2255-64; Cardiovasc Res. 2007; 73:470-80; Curr Drug Targets. 2007 December; 8(12): 1288-96).

For example, advanced human coronary artery lesions can have more apoptotic cells outside of lesional phagocytes than a control tissue, human tonsil, where most of the apoptotic cells are inside phagocytes (Arterioscler Thromb Vasc Biol. 2005; 25: 1256-61). Moreover, gene targeting of extracellular molecules that have been shown to bridge phagocytes to apoptotic cells in vitro, namely, complement C1q and lactadherin, promotes apoptotic cell accumulation (Am J Pathol. 2007; 170:416-26) and a vulnerable plaque phenotype in mouse atherosclerotic lesions (Circulation. 2007; 115:2168-77). A link between defective efferocytosis and accelerated atherosclerosis in hyperlipidemic mice lacking Fas ligand also exists (J Exp Med. 2004; 199:1121-31). Ldlr−/− mice lacking transglutaminase-2 (TG2), which has been implicated in efferocytosis, have increased atherosclerosis (Arterioscler Thromb Vasc Biol. 2006; 26:563-9). However, the human study was correlative, not causative, and C1q, lactadherin, Fas ligand, and TG-2 can have other important roles besides those in efferocytosis.

MerTK (also known as Eyk, Nyk, and Tyro-12) is a tyrosine kinase receptor for the phosphatidylserine-binding protein Gas6, which bridges apoptotic cells to phagocytes (J Biol Chem. 1996; 271:30022-7; Nature. 2001; 411:207-11). MerTK tyrosine kinase can signal through a pathway involving αvβ5 integrin, culminating in polymerization of the phagocyte cytoskeleton during apoptotic cell internalization (Nature. 2001; 411:207-11). In-vivo, apoptotic thymocyte removal is defective in mice carrying a tyrosine-defective MerTK (Mertk^{KD}) (Nature. 2001; 411:207-11; J Exp Med. 2002; 196:135-40).

In an in-vitro study, showed that macrophages from Mertk^{KD} mice have a defect in the ingestion of macrophages rendered apoptotic by cholesterol loading, which is a model of advanced lesional macrophage death. (J Biol Chem. 2006; 281:6707-17). We hypothesized that if the MerTK receptor functions similarly in advanced atherosclerotic lesions as it does in vitro, then defective MerTK may promote advanced lesional macrophage apoptosis and plaque necrosis. To test this hypothesis, Mertk^{KD} mice were crossed onto the Apoe−/− background and these mice and Apoe−/− control mice were fed a Western-type high-cholesterol diet for 10 or 16 wks. The plaques of the Mertk^{KD};Apoe−/− mice had a significant increase in TUNEL-positive apoptotic cells and, at the 16-wk timepoint, more plaque necrosis. While future studies will be needed to define precisely the mechanism of MerTK action in advanced lesions, these findings identify a new molecule that plays a protective role against plaque progression.

Methods

Animals and Diets

Mertk^{KD} mice on the C57BL6/J were bred onto the Apoe−/− C57BL6/J background to generate Mertk^{KD};Apoe−/− breeding pairs. Starting at 8 wks of age, progeny from these breeding pairs were fed a high-fat (21.2%), high-cholesterol (0.2%) Western-type diet (Catalog #TD88137) from Harlan Teklad (Madison, Wis.) for 10 weeks or 16 weeks. All animals were housed and cared for according to NIH and IACUC guidelines in a barrier facility at Columbia University Medical Center, New York, N.Y.

Plasma Lipid Analysis

At the end of the study, the mice were fasted overnight and then weighed, anesthetized using isoflurane, and euthanized. Blood was collected from the left ventricle. Total plasma cholesterol was measured with a commercially available kit from Wako. Plasma lipoprotein profiles were determined by fast performance liquid chromatography (FPLC) gel filtration on a Superose 6 column at a flow rate of 0.2 ml per minute. Eluted fractions were assayed for cholesterol using the Wako kit.

Atherosclerotic Lesion Analysis

Mouse hearts were perfused in-situ with saline, removed, and fixed in 10% formalin. Aortic roots were placed in biopsy cassettes, processed in a Leica tissue-processing machine, and embedded in paraffin blocks. Sections were cut serially at 6-μm intervals from the aortic sinus and mounted on slides. Prior to staining, sections were deparaffinized in xylene and rehydrated in graded series of ethanol. For area measurements and morphometric analysis, the sections were stained with Harris' hematoxylin and eosin. Total intimal lesional area was quantified by averaging six sections that were spaced 30 μm apart, starting from the base of the aortic root. Images were viewed and captured with a Nikon Labophot 2 microscope and analyzed using Image Pro Plus software. Apoptotic cells in lesions were detected by TUNEL (Tdt-mediated dUTP nick end labeling) after proteinase K treatment, using the in-situ cell death detection kit, TMR red, from Roche. Nuclei were counterstained with Hoechst for 5 minutes, and the slides were viewed and imaged by fluorescent microscopy. Plaque necrosis was quantified by measuring the area of hematoxylin and eosin-negative acellular areas in the intima, as described previously (Proc Natl Acad Sci USA. 2003; 100: 10423-8).

Statistical Analysis

Data are displayed as mean ±S.E.M. For the 10-wk cohort, n=5 per group, and for the 16-wk cohort, n=10 per group. Statistically significant difference between values for the two groups of mice was determined using the Student's paired t-test.

Results

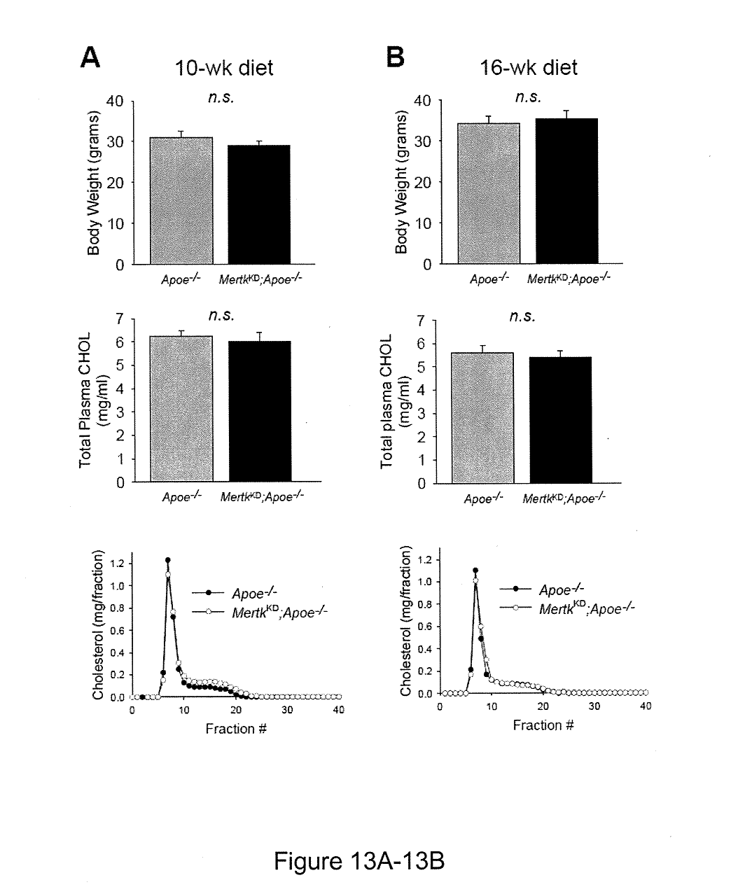

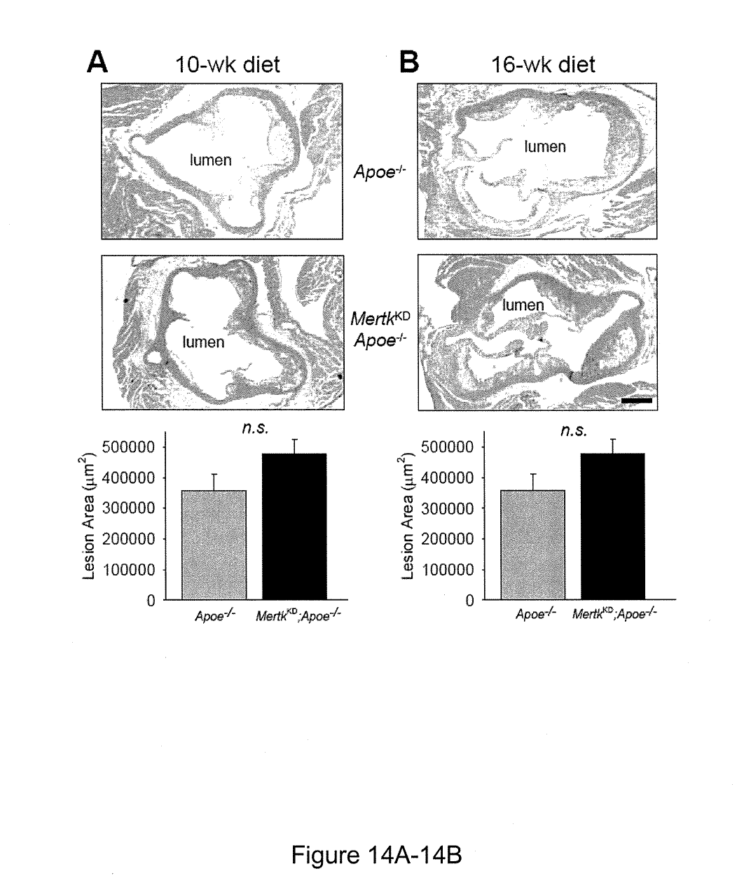

Mertk^{KD};Apoe−/− mice displayed no gross phenotypic differences compared with control Apoe−/− mice. After 10 or 16 wks on a Western-type diet, MerTK deficiency did not significantly affect overall body weight or plasma cholesterol parameters (FIG. 13). To determine the effect of MerTK deficiency on atherosclerosis, aortic roots from Apoe−/− and Mertk^{KD};Apoe−/− mice were sectioned and total atherosclerotic lesional area was measured at the end of the 10 & 16 week Western diets. The goals were to assess overall lesion size, which is affected primarily by processes involved in atherogenesis, and advanced plaque morphology, notably intimal cell apoptosis and plaque necrosis. Although defective MerTK could have a greater effect on advanced lesion morphology because of its role in the engulfment of macrophage rendered apoptotic by mechanisms likely to be more important in advanced lesions. (J Biol Chem. 2006; 281:6707-17), apoptosis and efferocytosis can affect both early lesion development and advanced plaque necrosis, (Arterioscler Thromb Vasc Biol. 2005; 25:2255-64; Cardiovasc Res. 2007; 73:470-80; Cell Metabolism. 2005; 1:201-13; Arterioscler Thromb Vasc Biol. 2005; 25: 174-9). The Mertk^{KD};Apoe−/− lesions showed a slight trend toward increased lesion area, but it was not statistically significant (FIG. 14). The fact that lesion area was not substantially affected by the MerTK mutation enabled a more focused analysis of advanced lesional morphology.

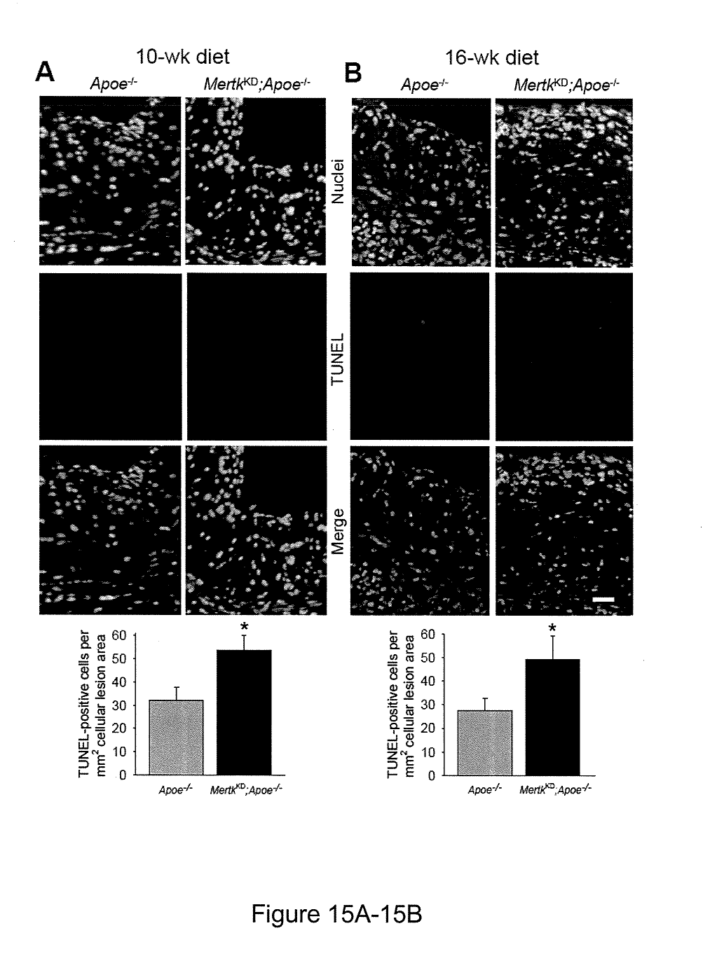

To detect apoptotic cells in the plaques, lesions from the two groups of mice were stained by the TUNEL method. Only TUNEL signal that colocalized with nuclei were counted. TUNEL-positive cells were of low frequency in Apoe−/− lesions (FIG. 15) (Cell Metab. 2007; 6:446-57). Lesions from both the 10- and 16-wk-fed Mertk^{KD};Apoe−/− mice exhibited a ˜70% increase in intimal TUNEL-positive cells relative to lesions from Apoe−/− mice (p<0.05).

The accumulation apoptotic cells in the intima in the setting of the Mertk^{KD} mutation was examined to determine if it is associated with increased plaque necrosis. Previous studies showed that intimal cell apoptosis precedes plaque necrosis (Lim W S et al, STAT1 is critical for apoptosis in macrophages subjected to endoplasmicreticulum stress in vitro and in advanced atherosclerotic lesions in vivo. Circulation. 2008).

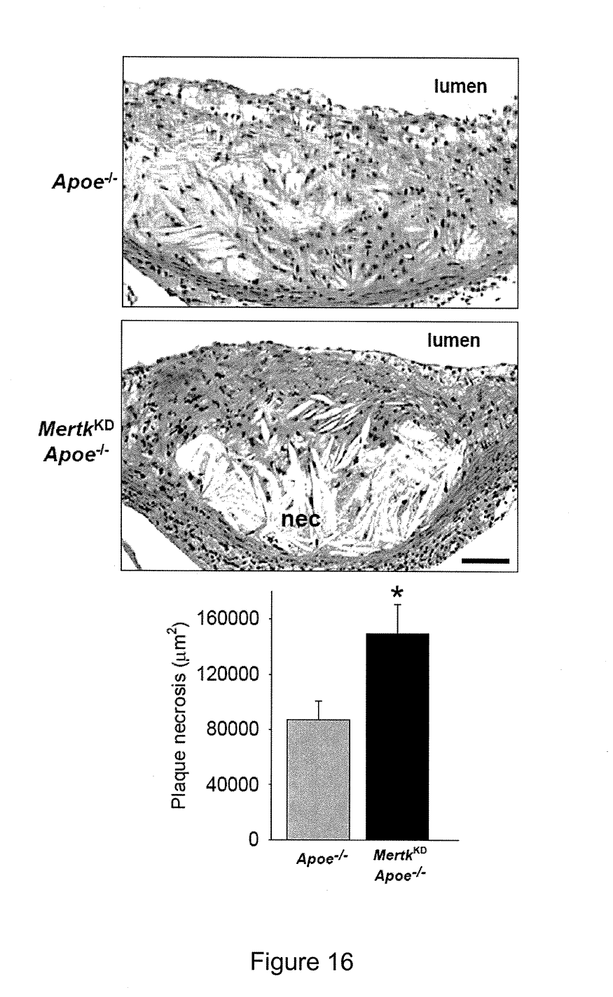

In the 10-wk-diet mice in the current study, plaque necrosis was not statistically different between the two groups of mice (data not shown). However, in the 16-wk-diet mice, plaque necrosis was increased by ˜75% in the Mertk^{KD};Apoe−/− lesions (p<0.05) (FIG. 16). Pending further in-vivo mechanistic studies, these data are show that non-cleared apoptotic macrophages, conferred by defective Mertk, can contribute, at least in part, to expansion of plaque necrosis in advanced atherosclerotic lesions.

Discussion

The data herein show that defective tyrosine kinase activity of the MerTK receptor leads to increased intimal cell apoptosis and plaque necrosis in the advanced lesions of Apoe−/− mice. This defect in MerTK does not affect plasma lipoproteins, and there was only a slight, non-statistically significant trend toward increased lesion size, indicating a more specific effect of the Mertk^{KD} mutation on advanced plaque morphology. This latter point is important, because it has been proposed that efficient efferocytosis of apoptotic macrophages in early lesions limits early lesion development (Arterioscler Thromb Vasc Biol. 2005; 25:2255-64; Cardiovasc Res. 2007; 73:470-80; Cell Metabolism. 2005; 1:201-13; Arterioscler Thromb Vasc Biol. 2005; 25:174-9).

The explanation for why defective MerTK did not have a greater effect than observed on the development of lesion area may show that the type of apoptotic macrophages in earlier lesions favor recognition by one or more of the many other efferocytosis receptors described in the literature (Chest. 2006; 129:1673-82). Indeed, MerTK had a non-redundant role in the efferocytosis of macrophages that were rendered apoptotic by a process—accumulation of unesterified cholesterol—that appears to be limited to advanced atherosclerotic lesions (J Biol Chem. 2006; 281:6707-17; J Clin Invest. 2002; 110:905-11).

Changes in advanced plaque morphology have been shown to be a more important indicator of human coronary atherosclerotic disease than overall lesion size (Annu Rev Med. 1993; 44:365-76). Given the known role of the tyrosine kinase activity of MerTK in efferocytosis both in vitro and in vivo, the findings from this study show that defective efferocytosis promotes advanced plaque progression (Arterioscler Thromb Vasc Biol. 2005; 25:2255-64; Curr Drug Targets. 2007 December; 8(12): 1288-96; Arterioscler Thromb Vasc Biol. 2005; 25:1256-61).

Because accelerated apoptosis could give rise to a similar plaque phenotype, presumably by saturating efferocytosis capacity in lesions, additional in-vivo mechanistic studies can be performed to show efferocytosis (Arterioscler Thromb Vasc Biol. 2005; 25:1256-61; J Exp Med. 2004; 199:1121-31; J Immunol. 2004; 173:6366-75) (Proc Natl Acad Sci USA. 2003; 100: 10423-8; Lim W S, Timmins J M, Seimon T A, Sadler A, Kolodgie F D, Virmani R, Tabas I. STAT1 is critical for apoptosis in macrophages subjected to endoplasmic reticulum stress in vitro and in advanced atherosclerotic lesions in vivo. Circulation. 2008.; Circulation. 2007; 116:2182-90.) The Mertk^{KD} mutation can also have other defects. For example, cells with this mutation have a heightened inflammatory response, (J Biol Chem. 2006; 281:6707-17; J Immunol. 1999; 162:3498-503) and if this effect occurs in atherosclerotic lesions, it can contribute to advanced plaque progression (Nature. 2002; 420:868-74). Defective efferocytosis in general is associated with increased inflammation, (Chest. 2006; 129:1673-82) and post-apoptotic cellular necrosis and inflammation may go hand-in-hand in terms of the consequences of decreased efferocytosis in advanced atherosclerosis. Efferocytosis activates cell-survival pathways in macrophages, (J Leukoc Biol. 2007; 82: 1040-50) and defective efferocytosis can be intimately linked with accelerated macrophage apoptosis. Whatever the results of future studies related to the mechanisms of MerTK in plaque progression, the data reveal the importance of a single molecule in a critical stage in advanced atherosclerosis, namely, expansion of the necrotic core.

A number of possibilities, including competitive inhibition of efferocytosis receptors by oxidized ligands, oxidant stress, and hypoxia may contribute to the mechanisms of defective efferocytosis in advanced lesions (Arterioscler Thromb Vasc Biol. 2005; 25:2255-64). Although genetic engineering can be used to get rid of lesions of a known efferocytosis receptor, MerTK can also be rendered dysfunctional in non-engineered lesions of an advanced stage.

The metalloproteinase TACE/ADAM17, which is expressed in advanced lesions, can cleave the extracellular domain of MerTK into a soluble receptor that can competitively bind to the Mertk/phosphatidylserine ligand Gas6 and inhibit phagocytosis of apoptotic cells (Blood. 2007; 109:1026-33; Atherosclerosis. 2006; 187:82-91). Mertk-mediated phagocytic activity can also be limited by the availability of Gas6. In this regard, vascular smooth muscle cells may be a major Gas6 source in lesions, (Trends Cardiovasc Med. 1999; 9:250-3; Electrophoresis. 2000; 21:3851-6) and vulnerable plaques have a deficiency of smooth muscles in areas of plaque disruption (Nat Med. 2006; 12:1075-80). Thus, the genetic manipulation described in this example can mimic an event that naturally occurs in very advanced lesions. Almost all humans in industrialized societies have evidence of atherosclerosis by the time they reach their teens or twenties (Circulation. 2007; 116:1832-44). Although most of these lesions are of the so-called “benign” type, meaning they lack the features of vulnerable plaques, (J Am Coll Cardiol. 2007; 50:940-9) those lesions that become necrotic and acquire other vulnerable traits wreak havoc in terms of the toll of ischemic vascular disease (J Am Coll Cardiol. 2007; 50:940-9; N Engl J Med. 1997; 337:1360-9). Moreover, there is an accelerating epidemic of diabetes-associated atherothrombotic vascular disease, (J Am Coll Cardiol. 2005; 46: 1225-8) and diabetic lesions are characterized by very large necrotic cores (Can J Cardiol. 2006; 22 Suppl B:81B-4B). For this reason, identifying specific molecular targets and processes that affect advanced plaque necrosis is critical. Measures to enhance efferocytosis in general, or MerTK function in particular, may lead to novel strategies to prevent acute atherothrombotic vascular disease (Arterioscler Thromb Vasc Biol. 2005; 25:2255-64; Curr Drug Targets. 2007 December; 8(12): 1288-96; Arterioscler Thromb Vasc Biol. 2005; 25:1256-61).

### Example 16

### Interleukin-4 Promotes Phagocytosis by Alternative Activation

Macrophage activation can depend on interferon-γ and a cytokine network involving interleukin-12 (IL-12) and IL-18, which are produced by antigen-presenting cells (APCs).

Activation of macrophages can also occur by cytokines that are produced in allergic, cellular and humoral responses to parasitic and extracellular pathogens (Nat Rev Immunol. 2003 January; 3(1):23-35). Exemplary cytokines capable of inducing alternative activation of cytokines include IL-4 and IL-13. IL-4 and IL-13 can up-regulate expression of the mannose receptor and MHC class II molecules by macrophages, which stimulate endocytosis and antigen presentation

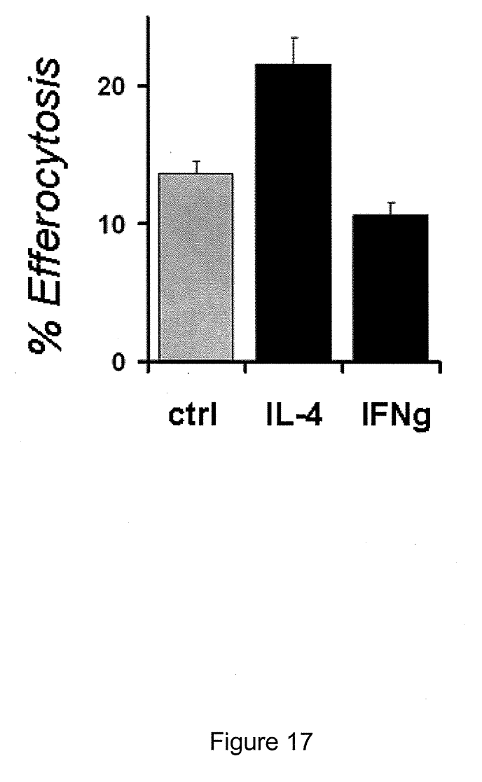

Treating phagocytes with compounds that promote an activation process called “alternative activation,” for example, interleukin-4, promotes the ability of these phagocytes to engulf apoptotic macrophages (FIG. 17). Thus, drugs that mimic this effect can promote the uptake of apoptotic macrophages in advanced atherosclerotic lesions (see in allergic, cellular and humoral responses to parasitic and extracellular pathogens (see Nat Rev Immunol. 2003 January;3(1):23-35).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying an enhancer of phagocytic clearance of free cholesterol-induced (FC-induced) macrophages, the method comprising
(a) labeling an FC-induced apoptotic macrophage;
(b) culturing the FC-induced apoptotic macrophage in the presence of phagocytes and in the presence of a test compound, thereby providing a test sample; and
(c) determining the amount of a label present in the phagocytes in the test sample, wherein, an increase in the amount of the label in the phagocytes in the presence of the test compound compared to the amount of the label present in the phagocytes cultured with FC-induced apoptotic macrophages in the absence of the test compound indicates that the compound is an enhancer of phagocytic clearance of a FC-induced apoptotic macrophages.

2. A method for identifying an enhancer of phagocytic clearance of free cholesterol-induced (FC-induced) macrophages, the method comprising
(a) labeling an FC-induced necrotic macrophage;
(b) culturing the FC-induced necrotic macrophage in the presence of phagocytes and in the presence of a test compound, thereby providing a test sample; and
(c) determining the amount of a label present in the phagocytes in the test sample, wherein, an increase in the amount of the label in the phagocytes in the presence of the test compound compared to the amount of the label present in the phagocytes cultured with FC-induced necrotic macrophages in the absence of the test compound indicates that the compound is an enhancer of phagocytic clearance of a FC-induced necrotic macrophages.

3. A method for identifying an enhancer of phagocytic clearance of free cholesterol-loaded macrophages, the method comprising
(a) labeling a free cholesterol-loaded macrophage;
(b) culturing the free cholesterol-loaded macrophage in the presence of phagocytes and in the presence of a test compound, thereby providing a test sample; and
(c) determining the amount of a label present in the phagocytes in the test sample, wherein, an increase in the amount of the label in the phagocytes in the presence of the test compound compared to the amount of the label present in the phagocytes cultured with free cholesterol-loaded macrophages in the absence of the test compound indicates that the compound is an enhancer of phagocytic clearance of a free cholesterol-loaded macrophages.

4. The method of claim 3, wherein the free cholesterol-loaded macrophage is a foam cell.

5. The method of claim 1, wherein the phagocytes are derived from peritoneal macrophages.

6. The method of claim 1, wherein the FC-induced apoptotic macrophage is labeled with calcein-AM.

7. The method of claim 1, wherein the FC-induced apoptotic macrophage is labeled with annexin V.

8. The method of claim 2, wherein the FC-induced necrotic macrophage is labeled with annexin V.

9. The method of claim 2, wherein the FC-induced necrotic macrophage is labeled with propidium iodide.

10. The method of claim 1, wherein the apoptotic macrophage is induced by contacting the macrophage with acetyl-low density lipoprotein (acetyl-LDL) and an acyl-coenzyme A:cholesterol acyltransferase (ACAT) inhibitor.

11. The method of claim 2, wherein the necrotic macrophage is induced by contacting the macrophage with acetyl-low density lipoprotein (acetyl-LDL) and an acyl-coenzyme A:cholesterol acyltransferase (ACAT) inhibitor.

12. The method of claim 2, wherein the cholesterol-loaded macrophage is induced by contacting the macrophage with acetyl-low density lipoprotein (acetyl-LDL) and an acyl-coenzyme A:cholesterol acyltransferase (ACAT) inhibitor.

13. The method of any of claim 10-12, wherein the ACAT inhibitor is 58035 ACAT inhibitor.

14. The method of any of claim 1-3, wherein the increase in the amount of label present in the phagocytes of the test sample compared to the amount of label in the macrophages cultured in the absence of the test compound is at least 10%, 20%, 25%, 30%, 50%, 75%, 90%, or 100% greater than the amount of label in the macrophages cultured in the absence of the test compound.

15. The method of any of claim 1-3, wherein the macrophages of (b) are cultured in the presence of a test compound and a statin, and the amount of label present in the phagocytes in the test sample is compared to the amount of label present in the phagocytes in the absence of the test compound and in the presence of the statin.

16. A method for promoting clearance of apoptotic macrophages from advanced atherosclerotic lesions, the method comprising contacting an atherosclerotic lesion with a compound that promotes clearance of apoptotic macrophages.

17. The method of claim 16, wherein the compound is a lipoxin, a lipoxin analog, a compound that stimulates lipoxin synthesis or activity, an annexin-1, an apolipoprotein E, a RhoA inhibitor, a RhoA kinase inhibitor, a thiazolinedione, interleukin-4, interleukin-13, a corticosteroid, eotaxin, yeast cell wall extract, P1-glucan, acemannan, tuftsin, a C1qRp ligand, an activator of 11-beta-hydroxysteroid dehydrogenase, a CCAAT/enhancer binding protein alpha, and inhibitor of farnesylation, an inhibitor of geranylgeranylation, or a compound that inhibits expression or activity of Cdc44.

18. The method of claim 16, wherein the method further comprises contacting the atherosclerotic lesion with a statin.

19. A method for treating atherosclerosis or inhibiting the development of atherosclerosis in a subject, the method comprising administering to the subject a compound that enhances macrophage phagocytosis.

20. A method for treating a subject at risk of having or having an atherosclerotic lesion, the method comprising administering to the subject a pharmaceutically effective amount of a compound that promotes clearance of apoptotic macrophages from advanced atherosclerotic lesions.

21. A method for treating a subject at risk of having or having an atherosclerotic lesion, the method comprising administering to the subject a pharmaceutically effective amount of a compound that promotes clearance of necrotic macrophages from advanced atherosclerotic lesions.

22. A method for treating a subject at risk of having or having an atherosclerotic lesion, the method comprising administering to the subject a pharmaceutically effective amount of a compound that promotes clearance of cholesterol-loaded macrophages from advanced atherosclerotic lesions.

23. The method of any of claim 19-22, wherein the compound comprises an annexin-1.

24. The method of any of claim 19-22, wherein the compound is an apolipoprotein E.

25. The method of any of claim 19-22, wherein the compound is interleukin-4 or interleukin-13.

26. The method of any of claim 19-22, wherein the compound is a peptidomimetic, a truncation product, or a fragment of an annexin-1, a lipoxin, or an apoplipoprotein E.

27. The method of any of claim 19-22, wherein the compound is a histidine-rich glycoprotein (HRG).

28. The method of any of claim 19-22, wherein the compound inhibits a RhoA or a RhoA kinase.

29. The method of claim 28, wherein the compound is fasudil or Y-27632.

30. The method of any of claim 19-22, wherein the compound is a thiazolinedione, a yeast cell wall extract, P1-glucan, acemannan, tuftsin, a C1qRp ligand, an activator of 11-beta-hydroxysteroid dehydrogenase, a CCAAT/enhancer binding protein alpha, and inhibitor of farnesylation, an inhibitor of geranylgeranylation, or a compound that inhibits expression or activity of Cdc44.

31. The method of any of claim 19-22, wherein the subject is characterized as having a history of heart disease, having diabetes, having atherosclerosis, or any combination thereof.

32. The method of any of claim 19-22, wherein the compound is a lipoxin.

33. The method of any of claim 19-22, wherein a statin is administered to the subject.

34. A composition comprising an enhancer of phagocytic clearance of apoptotic macrophages (a phagocyte enhancer compound) and a pharmaceutically acceptable excipient.

35. The composition of claim 34, wherein the phagocyte enhancer compound is identified using the method of any of claim 1.

36. The composition of claim 34, wherein the phagocyte enhancer compound is identified using the method of any of claim 2.

37. The composition of claim 34, wherein the phagocyte enhancer compound is identified using the method of any of claim 3.

38. The composition of claim 34, further comprising a statin.

39. A kit comprising a composition comprising an enhancer of phagocytic clearance of FC-induced apoptotic macrophages identified using the method of claim 1, and instructions for use.

40. A kit comprising a composition comprising an enhancer of phagocytic clearance of FC-induced necrotic macrophages identified using the method of claim 2, and instructions for use.

41. A kit comprising a composition comprising an enhancer of phagocytic clearance of free cholesterol-loaded macrophages identified using the method of claim 3, and instructions for use.
